(19) United States
(12) Patent Application Publication
(10) Pub. No.: US 2024/0168009 A1
(43) Pub. Date: May 23, 2024

(54) WEARABLE DEVICE FOR DETECTING SUBSTANCE ABUSE AND LOCATION

(71) Applicant: Robert Frank NIENHOUSE, Hinsdale, IL (US)

(72) Inventor: Robert Frank NIENHOUSE, Hinsdale, IL (US)

(21) Appl. No.: 18/513,044

(22) Filed: Nov. 17, 2023

Related U.S. Application Data
(60) Provisional application No. 63/426,100, filed on Nov. 17, 2022.

(51) Int. Cl. G01N 33/497; A61B 5/00; H04W 4/02

(52) U.S. Cl. CPC G01N 33/497; A61B 5/6831; A61B 5/7455; H04W 4/02

(57) ABSTRACT

A wearable device worn around a wrist or ankle of a test subject includes advanced GNSS functionality to track a location of the test subject, and detects abuse of a substance by the test subject. The wearable device includes an internal chamber with a control circuit, a substance detection sensor, a heater, and a pressure sensor. Optionally, the wearable device includes a fan to remove residual substance from the substance detection sensor and/or to move air from an air cavity between the wearable device and skin of the test subject into the substance detection sensor, which detects an amount of substance in the air. The control circuit detects when the wearable device is removed, measures resistance of the test subject's skin to detect when a membrane is inserted between the wearable device and the skin, and measures air pressure to determine when apertures are blocked by a foreign substance.

### RELATED APPLICATION

This application claims priority to U.S. Patent Application Ser. No. 63/426,100, titled “Wearable Device for Detecting Substance Abuse,” filed Nov. 17, 2122, which is incorporate in its entirety by reference herein.

### BACKGROUND

Conventional testing for substance abuse by a test subject requires capturing a breath sample from the test subject at intervals. Such testing requires the test subject to carry and operate the testing device on a given schedule. Thus, testing can be unreliable.

### SUMMARY

One aspect of the present embodiments includes the realization that an abused substance may be exuded through a person's skin into the air. Accordingly, substance abuse may be detected by sampling the air next to the skin of a test subject. The present embodiments take advantage of this realization by providing a wearable device (e.g., a watch type device) that includes a substance detection sensor such as a fuel cell capable of detecting the abused substance (e.g., alcohol) that is worn on a wrist or ankle of the test subject.

Another aspect of the present embodiments includes the realization that a substance detection sensor such as a fuel cell may provide a low-level reading from residual substance from a previous reading and sometimes caused when there is insufficient air flow between readings. Certain embodiments solve this problem by including a fan/micro-fan/micro-pump that is activated to increase air flow through the substance detection sensor to remove any residual substance and thereby provide more accurate and reliable readings.

Another aspect of the present embodiments includes the realization that a test subject may remove or tamper with the wearable device to prevent it detecting substance abuse (lack of compliance). Certain embodiments solve this problem by detecting when the wearable device is removed from the test subject's wrist or ankle by detecting when a strap of the wearable device is opened or cut. Further, a skin-side of the wearable device may include skin sensors (such as very low current electrical resistance skin contacts) that are used to measure connection of the wearable-device with the skin of the test subject and can thereby detect when a foreign substance, such as a clear film or other style of membrane, is inserted between the wearable device and the skin to prevent detection of the abused substance. In certain embodiments, the wearable device includes a heater that may be activated to change the humidity level within the device and air flow blockage sensors (e.g., air pressure sensors) to detect when apertures, used to receive air adjacent the test subject's skin, are blocked by a foreign substance (e.g., gel, paste, or other semi-liquid substance) that may not be detectable by the skin contacts.

Another aspect of the present embodiments includes the realization that conventional global navigation satellite system (GNSS) locationing has limited accuracy and suffers from signal degradation due to atmospheric conditions and building effects that result in drift of the determined location that prevents detection of stationary conditions. The present embodiments solve this problem by using an algorithm that processes the GNSS location data and other sensor data together to detect when a test subject is changing location, and when the test subject is not changing location. Advantageously, by determining when the test subject is stationary, a wearable device may conserve battery power, such as by not determining GNSS locations until other sensors indicate that the test subject is moving sufficiently to change location.

In certain embodiments, the techniques described herein relate to a wearable device for detecting an abused substance, including: a main body forming an internal chamber and having a first recessed area and a second recessed area on a skin-side of the main body, the main body forming a plurality of first apertures between the first recessed area and the internal chamber; a substance detection sensor located within the internal chamber; a strap attached to the main body for securing the wearable device to a wrist or ankle of a test subject; and a control circuit including a processor and memory storing machine-readable instructions that, when executed by the processor, cause the control circuit to: determine a first substance level value from an output of the substance detection sensor indicative of a level of the abused substance in air flowing through the internal chamber; and send the first substance level value to an external server.

In certain embodiments, the techniques described herein relate to a method for detecting substance compliance using a wearable device positioned on a wrist or ankle of a test subject, including: continuously detecting, by a substance detection sensor located in an internal chamber of the wearable device, a substance within air from a first recessed area, located between a skin-side of the wearable device and skin of the test subject, via a first aperture formed between the internal chamber and the first recessed area; determining a substance level value from an output of the substance detection sensor; and sending the substance level value to an external server when the substance level value indicates a level of the abused substance that is not in compliance.

In certain embodiments, the techniques described herein relate to an annunciating tracking device, including: a main body; a strap coupled with the main body; a clasp for securing the strap to itself to attach the annunciating tracking device to a test subject; a speaker located with the main body; and a control circuit positioned within the main body and having: a global navigation satellite system (GNSS) receiver; a long-range transceiver; a processor; and memory storing (a) one or more of first geographic coordinates defining an inclusion zone and second geographic coordinates defining an exclusion zone, and (b) machine-readable instructions that when executed by the processor cause the control circuit to: activate, at first intervals, the GNSS receiver to determine a current location of the annunciating tracking device; determine a location violation when the current location is outside the inclusion zone or the current location is within the exclusion zone; and output, via the speaker, a prerecorded or synthesized message with instructions for a person wearing the annunciating tracking device to correct the location violation.

### BRIEF DESCRIPTION OF THE FIGURES

FIG. 1 shows one example wearable device for detecting substance abuse, in embodiments.

FIGS. 2 and 3 show one example wearable device that may represent wearable device of FIG. 1, in embodiments.

FIG. 4 is a perspective view of the wearable device of FIGS. 2 and 3.

FIGS. 5 and 6 are schematics showing a wearable device that is similar to wearable device of FIGS. 2 and 3 but featuring an alternative channel design, in embodiments.

FIGS. 7A and 7B are perspective views of the wearable device of FIG. 1 showing a strap and clasp, in embodiments.

FIGS. 8A-8B are cross-sectional schematics showing example operation of the clasp of FIGS. 7A and 7B, in embodiments.

FIG. 8C shows a fastener of clasp in further example detail, in embodiments.

FIGS. 9 and 10 are schematics showing one example wearable device that is similar to the wearable devices of FIGS. 2, 3, 5 and 6, but featuring an alternative channel design and internal component positioning, in embodiments.

FIG. 11 shows an internal view of a bottom portion of the wearable device of FIGS. 9 and 10 where a top portion of the main body has been removed, in embodiments.

FIG. 12 is a flowchart illustrating one example sleep method for conserving battery power within a wearable device, in embodiments.

FIG. 13 is a flowchart illustrating one example method for detecting substance abuse using the wearable device of FIGS. 1, 2, 3, 5 and 6, in embodiments.

FIG. 14 is a flowchart illustrating one example method for removing residual substance from a substance detection sensor after a detected substance level has exceeded a threshold value, in embodiments.

FIG. 15 is a flowchart illustrating one example method for detecting when operation of the device of FIGS. 2, 5, and 9, respectively, is compromised, in embodiments.

FIG. 16 is a flowchart illustrating one example method for detecting when the wearable device of FIGS. 2 and 5 is removed, in embodiments.

FIG. 17 is a schematic illustrating one example annunciating tracking device for monitoring movement of a person, in embodiments.

FIG. 18 is a cross-sectional schematic diagram illustrating the annunciating tracking device of FIG. 17 in further example detail, in embodiments.

FIG. 19 is a flowchart illustrating one example method for detecting and announcing location violations by the annunciating tracking device of FIG. 17, in embodiments.

FIG. 20 is a flowchart illustrating another example method for detecting and announcing location violations by the annunciating tracking device of FIG. 17, in embodiments.

FIG. 21A is a cross-sectional diagram of a wearable device during manufacturer illustrating connection of a strap to a main body of the wearable device, in embodiments.

FIG. 21B is a perspective view showing connection of the strap to the main body of the wearable device of FIG. 21A, in embodiments

### DETAILED DESCRIPTION OF THE EMBODIMENTS

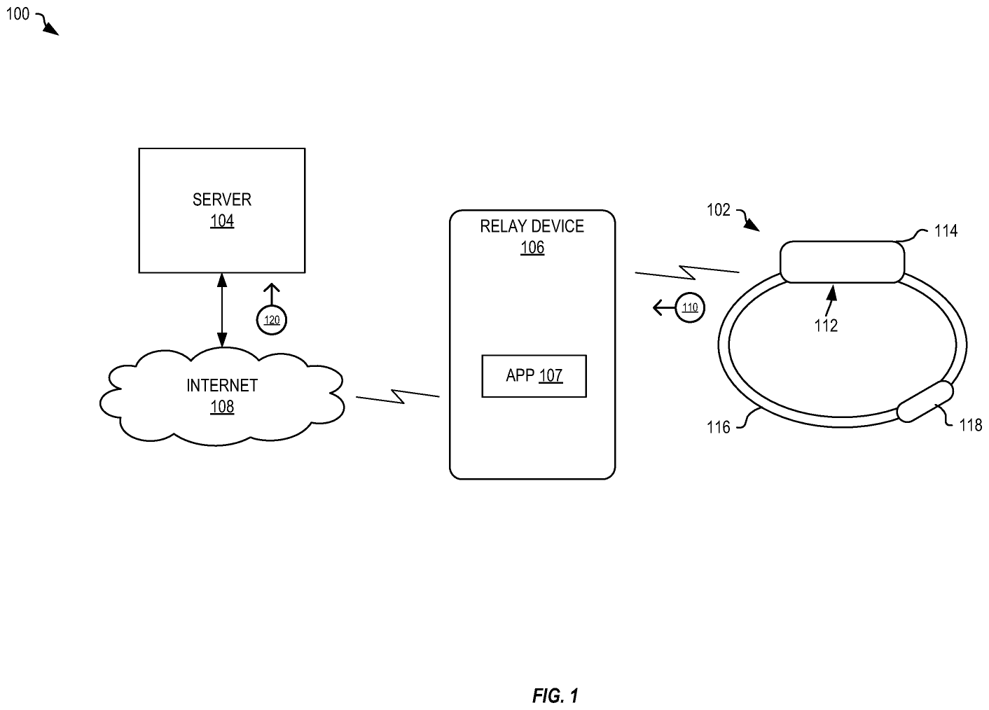

FIG. 1 shows one example wearable device 102 for detecting substance abuse. Device 102 forms part of a substance compliance/abuse monitoring system 100 that provides continuous monitoring for substance abuse by a test subject wearing device 102. In the following embodiments and examples, device 102 is a watch worn on a wrist or ankle of the test subject to detect alcohol abuse. However, other form factors and abuse substances may be implemented without departing from the scope hereof.

Device 102 has a main body 114, a strap 116, and a clasp 118. Device 102 detects the presence of alcohol in air at a skin-side 112 of main body 114 when positioned at a surface of the test subject's skin. Device 102 wirelessly communicates a status message 110 (e.g., test readings including a level of an abused substance, battery status, tamper detection, device removal, and so on) to a relay device 106 (e.g., a smartphone, a vehicle interface, a table computer, a laptop computer, etc.) and relay device 106 sends the received information to a server 104 (e.g., an external or remote server or a cloud service) via the Internet 108 (e.g., using one or more of a cellular connection, Wi-Fi connection, etc.) as a server message 120. Server message 120 may include substantially the same information as status message 110. In certain embodiments, device 102 communicates with relay device 106 using the Bluetooth protocol. For example, where relay device 106 is a smartphone, device 102 may send status message 110 to an app 107 running on relay device 106 when relay device 106 is in wireless communication range of device 102. Where relay device 106 is not in communication range of device 102, device 102 may buffer any generated status messages 110 and forward the buffered status messages 110 to app 107 when relay device 106 is available for communication (e.g., within wireless communication range, turned on, etc.). Where relay device 106 is unable to send server message 120 to server 104, app 107 may buffer information of status messages 110 within relay device 106 and send the buffered information to server 104 as server messages 120 when communication is available. Device 102 generates status messages 110 at a low frequency (e.g., one status message per hour) when the test subject is in compliance with no substance abuse (e.g., measured valued below a predefined threshold), and no device tampering (e.g., no detected removal or inhibited operation of device 102). Accordingly, app 107 sends server message 120 as a heartbeat message every hour, thereby minimizing network traffic that the test subject may have to pay for. However, where device 102 detects non-compliance through substance abuse (e.g., measured values at or above a predefined threshold), and/or device tampering (e.g., device 102 detecting removal and/or aperture blockage), device 102 may generate and send status message 110 more frequently to app 107, which may therefore send server message 120 to server 104 more frequently. In one example of operation, where device 102 determines that a test reading (e.g., taken once a minute, once every five minutes, and so on), is above a predefined threshold, device 102 may generate and send status messages 110 to relay device 106 for each test reading. Similarly, device 102 may send tampering information in status message 110 immediately that tampering is detected.

In certain embodiments, relay device is a vehicle interface that controls operation of a motor vehicle (e.g., a car or a truck), where device 102 and the vehicle interface interact to enable (or disable) operation of the vehicle. In embodiments, the vehicle interface may require that the wearable device 102 report a negative test reading before operation of the vehicle is enabled. For example, where device 102 reports a positive test reading (e.g., an alcohol level above a predefined threshold), relay device 106 may disable operation of the vehicle and/or report the test reading to server 104. In certain embodiments, device 102 may determine when the test subject is positioned in the driver's seat by determining its proximity (e.g., based on signal strength) to a transceiver of the vehicle interface positioned at the steering wheel. For example, when the transceiver of the vehicle interface is mounted at the back of the steering wheel or on a column of the steering wheel, and device 102 determines that it is within a predefined distance of the transceiver, device 102 may determine that the test subject is the driver and not a passenger in the vehicle.

The use of relay device 106 has numerous advantages compared to device 102 transmitting status message 110 directly to a service provider, since relay device 106 allows use of a carrier network of the test subject, and further allows communication from server 104 to the test subject using relay device 106. Advantageously, this reduces the power requirement for wearable device 102 and reduces the size of wearable device 102 as compared to a device that transmits directly with cellular and/or Wi-Fi. Further, the use of app 107 running on relay device 106 (e.g., the smartphone) allows additional information (e.g., location information) to be collected and included within server message 120, and provides a more convenient interface, as compared to an interface of device 102, for interacting with the test subject. In certain embodiments, device 102 may also include a haptic device (e.g., a vibration generator) and/or a display screen that may be used to communicate with the test subject. In another embodiments, device 102 may also include a speaker, whereby device 102 may play recorded message and/or sounds to the test subject.

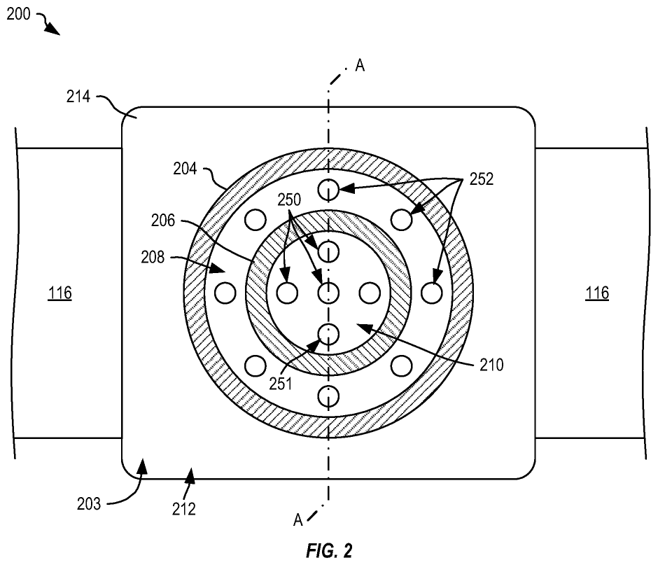

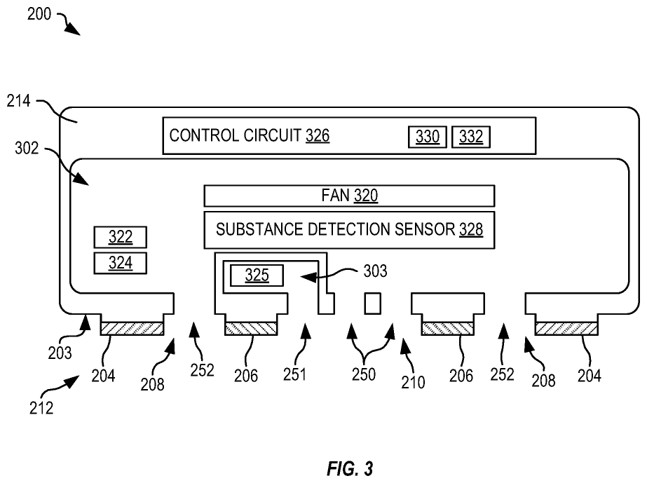

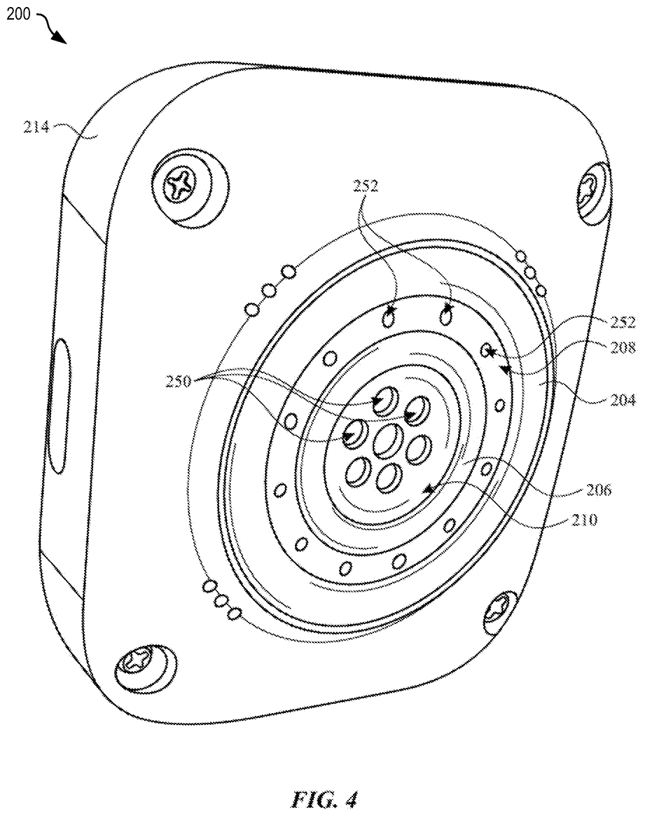

FIGS. 2 and 3 show one example wearable device 200 that may represent wearable device 102 of FIG. 1. FIG. 2 is a schematic showing a bottom view of a main body 214 of wearable device 200 and FIG. 3 is a cross section A-A through main body 214. FIG. 4 is a perspective view of a skin-side surface 212 of wearable device 200 of FIGS. 2 and 3 showing channels 208 and 210. FIGS. 2, 3, and 4 are best viewed together with the following description.

A first skin contact 204 and a second skin contact 206 (e.g., two separate contacts such as conductive rings, arcs or bars) are positioned at skin-side surface 212 of main body 214 and are raised from a bottom surface 203 of main body 214 and form an outer channel 208 (e.g., a first air cavity between the two rings or bars) and an inner channel 210 (e.g., a second air cavity within the inner ring). Main body 214 forms an internal chamber 302 that is open to ambient air only at bottom surface 203, a skin-side surface 212, via a plurality of outer apertures 252 within outer channel 208 and formed between bottom surface 203 and internal chamber 302, and a plurality of inner apertures 250 within inner channel 210 and formed between bottom surface 203 and internal chamber 302. In certain embodiments, outer apertures 252 may be formed at other positions on any side or the front of main body 214. Inner channel 210 is sized and shaped to provide an airspace adjacent to the test subject's skin that allows the abused substance (e.g., alcohol) to pass through the skin into air within the airspace. For example, inner channel 210 is circular with a depth of 1/16^{th} of an inch and a diameter of 9/16^{th} of an inch, and outer channel 208 is an annulus formed around inner channel 210 and has a depth of 1/16^{th} of an inch, an outer diameter of 1 and 3/16^{th} inches, and an inner diameter of ⅞^{th} of an inch. Skin contacts 204 and 206 may be metal (e.g., brass, stainless steel, and may be gold plated, etc.) and conduct electricity.

A substance detection sensor 328 (e.g., a transdermal diffusion fuel cell) is positioned within internal chamber 302 to receive air via apertures 250 and/or 252. For example, natural movement of the test subject causes device 102 to move (e.g., rock) against the test subject's skin, whereby the rocking movement may cause air (e.g., a transdermal sample from the test subject that is more diluted by ambient air) within inner channel 210 to flow through apertures 250 into chamber 302 and then out through apertures 252. A first side of substance detection sensor 328 may be fluidly connected with apertures 250 and a second side of substance detection sensor 328 is fluidly connected with apertures 252 such that air flowing between apertures 250 and 252 passes through substance detection sensor 328. Substance detection sensor 328 electrically connects (connections not shown for clarity of illustration) with a control circuit 326 also contained within main body 214. Control circuit 326 may include one or more of: a processor 330, memory 332 storing machine-readable instructions executable by processor 330, conditioning circuity, interface circuity, wireless communication circuity (e.g., Bluetooth, BLE, etc.), a watch display, and a battery. Control circuit 326 may include other components without departing from the scope hereof. Substance detection sensor 328 represents a fuel cell or solid-state sensor for detecting one or more of many different substances, elements, and compounds. For example, substance detection sensor 328 may represent a fuel cell for detecting alcohol, a solid-state sensor for detecting marijuana, and so on.

In the embodiment where substance detection sensor 328 is a fuel cell for detecting alcohol, a chemical reaction within substance detection sensor 328 generates an electrical current when alcohol is present in the air flowing through substance detection sensor 328, where an amount of the electrical current corresponds to an amount of alcohol present in the air. Accordingly, substance detection sensor 328 operates continuously to detect substance abuse. Control circuit 326 measures the electrical current from substance detection sensor 328 to determine a test reading indicative of the amount of abused substance (e.g., alcohol) in the air.

Humidity in air flowing through device 200 adversely effects operation of substance detection sensor 328 to detect the abused substance. Accordingly, skin-side surface 212 of main body 214 is designed to allow air not flowing through apertures 250/252 to disperse away from device 200, thereby reducing humidity between the test subject's skin and device 200. Advantageously, the amount of humidity that would otherwise collect and flow through apertures 250/252 is reduced, thereby reducing the adverse effects of humidity on substance detection sensor 328.

Channels 208 and 210 are a novel design that facilitate collection of air from above the skin of the test subject when wearable device 200 is worn and also prevent the test subject's skin from blocking apertures 250 and 252 that would prevent air flow. For example, without channels 208 and 210, the test subject's skin would block apertures 250 and 252 since bottom surface 203 would press against the skin, leaving no space for air flow in or out of apertures 250 or 252.

### Alternative Channel and Skin Contact Design

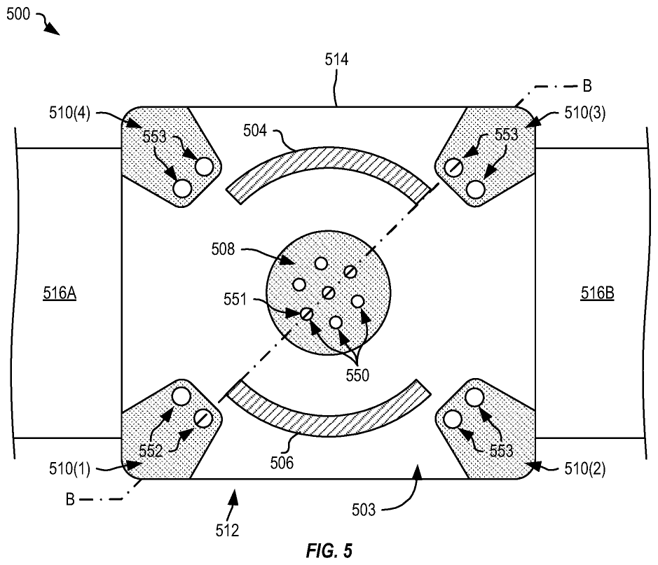

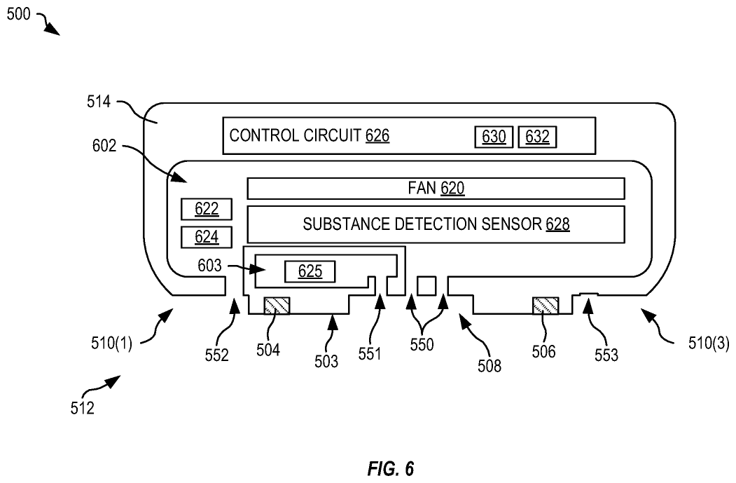

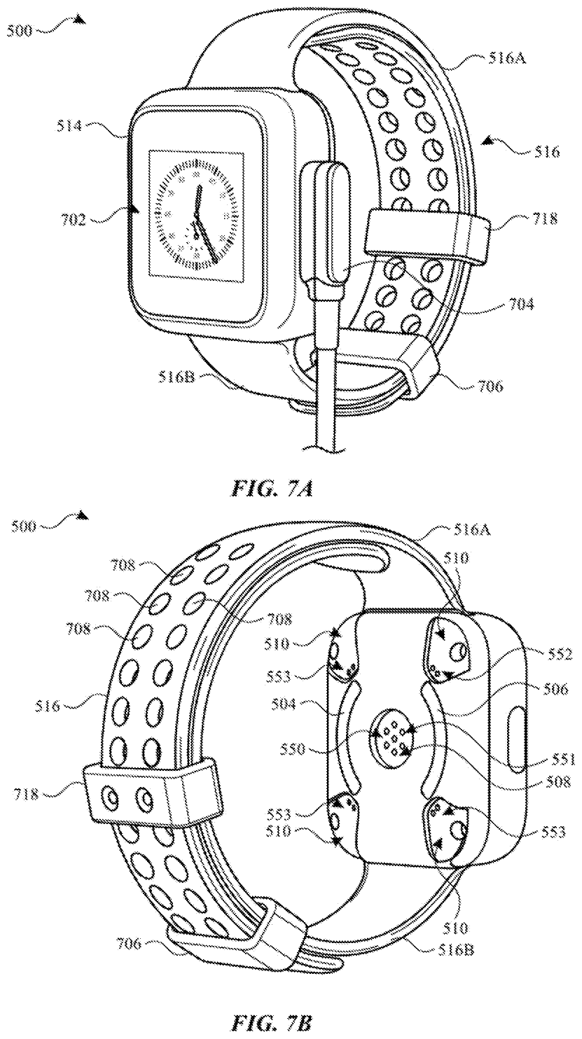

FIGS. 5 and 6 are schematics showing a wearable device 500 that is similar to wearable device 200 of FIGS. 2 and 3 but featuring an alternative channel design. Wearable device 500 may represent wearable device 102 of FIG. 1. FIG. 5 is a schematic showing a bottom view of a main body 514 of wearable device 500 and FIG. 6 is a cross section B-B through main body 514 of FIG. 5. FIGS. 7A and 7B show perspective views of wearable device 500 of FIGS. 5 and 6. FIGS. 5, 6, 7A, and 7B are best viewed together with the following description.

Wearable device 500 may include a time display 702 and associated time mechanism (e.g., mechanical, or digital) and may have a port for coupling with a charging cord 704; however, other embodiments of wearable device 500 may use wireless charging without departing from the scope hereof. A bottom surface 503 of main body 514 includes two arced skin contacts 504 and 506, an inner recessed area 508, and four corner recessed areas 510. Skin contacts 504 and 506 may be metal (e.g., brass, stainless steel, and may be gold plated, etc.) and conduct electricity. A plurality of inner apertures 550 are formed between inner recessed area 508 and an internal chamber 602 of main body 514 and a plurality of outer apertures 552 are formed between each corner recessed area 510 and internal chamber 602. As shown in FIG. 5, apertures 552 are formed within corner recessed area 510(1), and corner recessed areas 510(2)-(4) have blind holes 553, causing bottom surface 503, a skin-side surface 512, of main body 514 to appear symmetrical/balanced and thereby aesthetically pleasing. However, in certain embodiments one or more of blind holes 553 may be replaced by apertures that function substantially like one of apertures 552. In certain embodiments, apertures 552 may be formed at other positions, such as at any side or at a front of main body 514.

When wearable device 500 is worn, inner recessed area 508 forms an airspace adjacent to the test subject's skin, from where air flows through substance detection sensor 628 via inner apertures 550 and outer apertures 552. For example, natural movement of the test subject causes device 500 to move (e.g., rock) against the test subject's skin, whereby the rocking movement may cause air (e.g., a transdermal sample from the test subject) within inner recessed area 508 to flow through apertures 550 into chamber 602 and then out through apertures 552. Further, movement of ambient air around wearable device 500 draws the air sample out of chamber 602 through apertures 552 and away from wearable device 500, aided by corner recessed areas 510 being open to ambient air. Inner recessed area 508 is sized and shaped such that the airspace allows the abused substance (e.g., alcohol) to pass through the test subject's skin into air within the airspace. For example, inner recessed area 508 is circular with a depth of 3/64^{th} of an inch and a diameter of 7/16^{th} of an inch. Corner recessed areas 510 each provides an airspace adjacent to the test subject's skin. Particularly, corner recessed area 510(1) allows air to flow between internal chamber 602 and the external environment via apertures 552. For example, corner recessed areas 510 are approximately trapezoidal, have a depth of 1/16^{th} of an inch and an area similar to that of inner recessed area 508.

Inner recessed area 508, inner apertures 550, corner recessed areas 510, and outer apertures 552 are a novel design that facilitate collection of air from above the skin of the test subject when device 500 is worn and also prevent the test subject's skin from blocking apertures 550 and 552 that would prevent air flow. For example, without inner recessed area 508 and corner recessed areas 510, the test subject's skin pressing against bottom surface 503 would prevent air flow through apertures 550 and/or apertures 552.

Humidity in air flowing through device 500 adversely effects operation of substance detection sensor 628 to detect the abused substance. Accordingly, skin-side surface 512 of main body 514 is designed to include corner recessed areas 510 that allow air not flowing through apertures 550/552 to disperse away from device 500, thereby reducing humidity between the test subject's skin and device 500. Advantageously, the amount of humidity that would otherwise collect and flow through apertures 550/552 is reduced, thereby reducing the adverse effects of humidity on substance detection sensor 628.

Substance detection sensor 628 electrically connects (connections not shown for clarity of illustration) with a control circuit 626 also contained within main body 514. Control circuit 626 may include one or more of: a processor 630, memory 632 storing machine-readable instructions executable by processor 630, conditioning circuity, interface circuity, wireless communication circuity (e.g., Bluetooth, BLE, etc.), a watch display, and a battery. Control circuit 626 may include other components without departing from the scope hereof.

A chemical reaction within substance detection sensor 628 generates an electrical current when alcohol is present in the air flowing through substance detection sensor 628, where an amount of the electrical current corresponds to an amount of the abused substance (e.g., alcohol) present in the air. Control circuit 626 measures the electrical current from substance detection sensor 628 to determine the amount of abused substance in the air.

### Substance Detection Sensor

Substance detection sensor 328/628 is a transdermal diffusion fuel cell positioned within internal chamber 302/602 to receive air via apertures 250/550 and 252/552. Substance detection sensor 328/628 electrically connects (connections not shown for clarity of illustration) with control circuit 326/626 that may include one or more of conditioning circuity, interface circuity, wireless communication circuity, a watch display, and a battery. Control circuit 326/626 may include other components without departing from the scope hereof.

A chemical reaction within substance detection sensor 328/628 generates an electrical current when the abused substance (e.g., alcohol) is present in air flowing through substance detection sensor 328/628, where the amount of the electrical current generated by substance detection sensor 328/628 is proportional to the amount of alcohol present in the air. Control circuit 326/626 measures the electrical current to determine the amount of abused substance in the air.

### Skin Contacts

Prior art watch solutions that detect the watch (or similar device) is worn typically use an LED light source and a light sensor to detect reflectance of the LED light off a wearer's skin. When no reflectance is detected or when the reflectance does not contain readings similar to base readings determined by prior readings, the watch determines it is not being worn or a material has been placed between the device and the skin that prevents skin readings of insensible perspiration. One aspect of the present embodiments includes the realization that a test subject may attempt to prevent wearable device 200/500 from sensing air by placing a film (e.g., e.g., a clear film such as cellophane, or ither types of film or sheet such as paper) between wearable device 200/500 and their skin. The prior art LED light source and light sensor solution detects reflectance through or from the film, and therefore cannot discern whether the film is present. This would prevent the prior art watch from generating an alert when the test subject compromises operation of the wearable device by placing a film between the device and the test subject's skin. Advantageously, the present embodiments overcome this problem by measuring resistance of the test subject's skin that is in contact with the wearable device. For wearable device 200, first skin contact 204 and second skin contact 206 are electrically coupled to control circuit 326 that measures an electrical resistance of the skin between first skin contact 204 and second skin contact 206. For wearable device 500, arced skin contacts 504 and 506 are electrically coupled to control circuit 626 that measures an electrical resistance of the skin between arced skin contacts 504 and 506.

In one example of operation, at intervals (e.g., once per minute) control circuit 326/626 applies a small voltage across skin contacts 204/504 and 206/506 and measures a current flowing through the skin between the skin contacts. While wearable device 200/500 is worn correctly, the measured current remains within an expected range; however, when control circuit 326/626 determines that the measured current is outside the expected range, control circuit 326/626 determines that device 200/500 has been removed from the test subject's skin and/or a film has been placed between the test subject's skin and device 200/500. In another example of operation, control circuit 326/626 applies a small voltage continuously across skin contacts 204/504 and 206/506 and detects changes in a current flowing through the skin between the skin contacts indicative of device 200/500 being removed from the test subject's skin and/or a film being placed between the test subject's skin and device 200/500.

Since device 200/500 may momentarily decouple from the test subject's skin during natural movements made by the test subject, such momentary disconnections are not considered significant. For example, control circuit 326/626 may send an alert and/or notification to server 104 indicating that wearable device 200/500 is compromised. Accordingly, when the test subject attempts to use the film to stop wearable device 200/500 sensing alcohol, because the sensed electrical current changes, control circuit 326/626 may alert server 104 of the anomaly. In certain embodiments, control circuit 326/626 may send detected current values and/or detected changes thereto to server 104. Accordingly, server 104 is aware of tampering with wearable device 200/500 and/or removal of wearable device 200/500.

### Micro-Fan/Micro-Pump

As described above, the airflow through chamber 302/602 may occur naturally through apertures 250/550 and 252/552. One aspect of the present embodiments includes the realization that, unlike the prior art use of fuel cells in breathalyzers that rely upon an outward breath of a test subject to move air through the fuel cell, air above the test subject's skin may not flow sufficiently well through substance detection sensor 328/628 through natural motions of the test subject. Certain embodiments solve this problem by including a fan 320/620 (e.g., a micro-fan and/or a micro-pump) within internal chamber 302/602 to draw air through substance detection sensor 328/628 via apertures 250/550 and 252/552. Where sufficient air flows naturally through substance detection sensor 328/628, fan 320/620 is not included. Fan 320/620, when operated by control circuit 328/628 draws air in through apertures 250/550 and push air out through apertures 252/552. In certain embodiments, fan 320/620 is a micropump and the term fan 320/620 as used herein may refer to either a micropump or a fan. In one embodiment, fan 320/620 uses a piezoelectric motor (e.g., a thin membrane with a piezo electric crystal that is activated by a small electric current). In one example of operation, control circuit 326/626 provides an electrical signal to activate fan 320/620 and cause air to flow through substance detection sensor 328/628 via apertures 250/550 and 252/552, thereby moving air from above the skin of the test subject, through apertures 250/550, through substance detection sensor 328/628, and out through apertures 252/552. In embodiments where fan 320/620 has a reciprocating action, wearable device 200/500 may include one or more microvalves that aid flow of air in through apertures 250/550, through substance detection sensor 328/628, and out through apertures 252/552. In other embodiments, fan 320/620 has a rotational action of a propeller that moves air through in through apertures 250/550, through substance detection sensor 328/628, and out through apertures 252/552.

Wearable device 200/500 may be programmed with an alcohol threshold (e.g., X mg/l) and control circuit 326/626 reports an alcohol event when a test reading is greater than the alcohol threshold. Residual alcohol within fuel-cell based analyzers is a known problem and the residual alcohol typically causes alcohol events to be incorrectly reported requiring that those events need to be ignored at some point (e.g., at the server and/or when reporting). Residual alcohol in substance detection sensor 328/628 may result in a low level reading since substance detection sensor 328/628 reacts continuously to the presence of alcohol. Therefore, when an alcohol event is detected by wearable device 200/500, control circuit 326/626 operates fan 320/620 to remove any alcohol containing air from substance detection sensor 328/628, thereby ensuring subsequent evaluations are accurate. For example, control circuit 326/626 may operate fan 320/620 until a measured level indicated by substance detection sensor 328/628 is below the alcohol threshold.

### Pressure Sensor

Another aspect of the present embodiments includes the realization that a test subject may use a foreign substance (e.g., a gel or a similar substance) to clog/block apertures 252/552 and/or 250/550 to prevent substance detection sensor 328/628 from receiving air and thereby prevent device 200/500 from detecting substance abuse. The present embodiments solve this problem by detecting when apertures 252/552 and/or 250/550 are blocked. Wearable device 200/500 also includes a first pressure sensor 324/624 and an optional heater 322/622 (e.g., a resistive heating element) within internal chamber 302/602 that is accordingly fluidly connected to apertures 252/552. At intervals (e.g., once every 24 hours), control circuit 326/626 determines a first ambient air pressure within internal chamber 302/602 by reading pressure sensor 324/624. Control circuit 326/626 may activate heater 322/622, where included, for correct operation of pressure sensor 324/624. Wearable device 200/500 also includes a second pressure sensor 325/625 within a sub-chamber 303/603 that is formed by main body 214 to be fluidly connected with only one aperture 251/551 (e.g., one of apertures 250/550) and fluidically isolated from internal chamber 302/602. Proximity of sub-chamber 303/603 to substance detection sensor 328/628 alleviates the need for a second heater near second pressure sensor 325/625, since pressure sensor 325/625 benefits from heat generated by substance detection sensor 328/628. However, in certain embodiments, the second heater may be included for activation by control circuit 326/626 to heat second pressure sensor 325/625 for correct operation. Control circuit 326/626 reads a second air pressure from pressure sensor 325/625. When control circuit 326/626 determines that the first and second air pressures are the same or within a predefined threshold value of one another, there is no significant blockage of apertures 250/550 and 252/552 that would prevent air flow into substance detection sensor 328/628. When control circuit 326/626 determines that the first and second air pressures are significantly different (e.g., by at least the predefined threshold amount), control circuit 326/626 determines that apertures 250/550 and/or 252/652 may be blocked and control circuit 326/626 sends an alert and/or notification to server 104 indicative of tampering with operation of device 200/500. Advantageously, control circuit 326/626 detects when the test subject is attempting to compromise operation of wearable device 200/500 by blocking apertures 250/550 and/or 252/652 with a gel, paste, or other semi-liquid substance. Control circuit 326/626 may read pressure sensor 325/625 at a faster rate (e.g., more often) than reading of pressure sensor 324/624.

### Strap

A strap 116 of wearable device 102 (e.g., strap 516 of wearable device 500, FIG. 7A and 7B) has a material composition that that is electrically conductive (e.g., one or more of rubber, silicone, plastic, and polyurethane, in a formulation that incorporates a conductive element or compound, such as carbon black, throughout the strap). Control circuit 326/626 applies a small voltage to opposite ends of strap 516 (e.g., where strap portions 516A and 516B connect to opposite sides of main body 514) such that a small electrical current passes through strap 516 when clasp 718 secures the distal ends of strap 516 together. Control circuit 326/626 may detect when the small current through strap 516 changes (e.g., when strap 516 is cut or when clasp 718 is opened) and determine that wearable device 102/200/500 has been removed. Accordingly, control circuit 326/626 may monitor the small current through strap 516 for continuity and/or change to ensure wearable device 102/200/500 is not removed. For example, where control circuit 326/626 senses that the small current through strap 516 has changes, control circuit 326/626 determines that wearable device 102/200/500 is being tampered with, or is being, or has been, removed.

Prior art solutions to detect when a watch type device is removed may use an embed wire, light pipe, or other fiber optic, within a non-conductive strap, whereby current through the wire is measured to determine that the strap is closed and that the watch device has not been removed. However, these wires are unreliable and straps with embedded wires are more expensive to produce as compares strap 116. Advantageously, strap 516 and clasp 718 simplify manufacture, reduce cost, and improve reliability of wearable device 102/200/500.

FIGS. 7A and 7B also show a strap 516 and clasp 718 of wearable device 500, where strap 516 represents strap 116 of FIG. 1 and clasp 718 represents clasp 118. Strap 516 of wearable device 500 is formed of two strap portions 516A and 516B that each attach to a different side of main body 514. Each of strap portions 516A and 516B forms a plurality of holes 708 that may be overlapped and aligned such that clasp 718 joins strap portions 516A and 516B, whereby the selected overlap makes strap 516 adjustable and a one-size-fits-all design. Strap 516 may also include a loop 706 to hold strap portions 516A and 516B together.

### Clasp

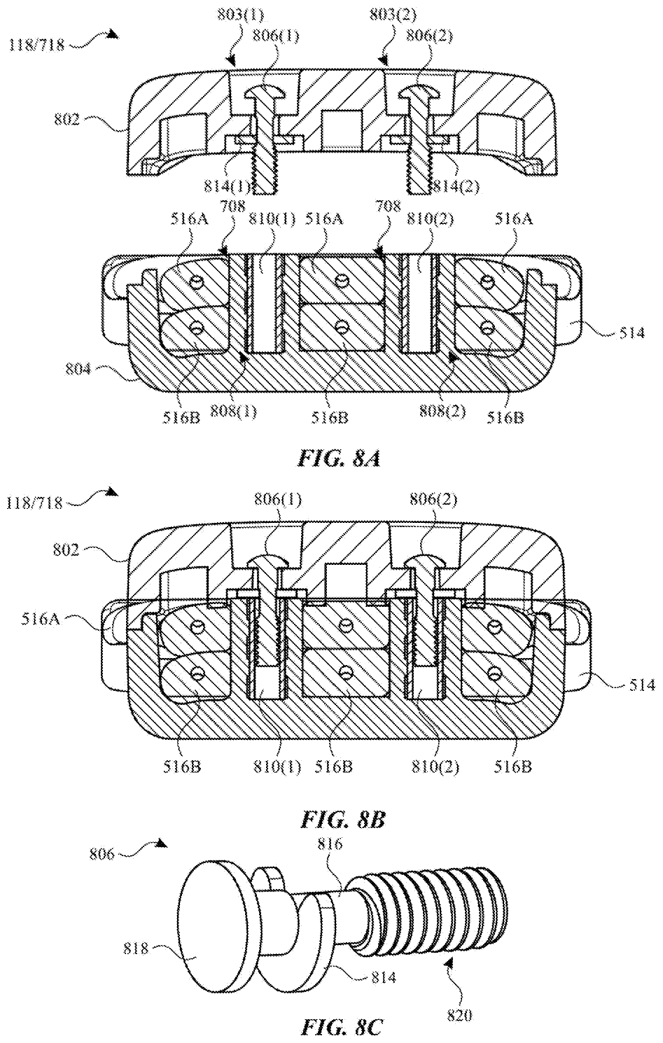

FIGS. 8A-8B are cross-sectional schematics showing example operation of clasp 718 of FIGS. 7A and 7B. FIG. 8C shows a fastener 806 of clasp 718 in further example detail. FIGS. 7A, 7B, 8A, 8B, and 8C are best viewed together with the following description.

Clasp 718 is a molded plastic material that separates into an outer part 802 and an inner part 804. The terms inner and outer are used for clarity of description with reference to FIGS. 8A and 8B and are not intended to be limiting, since clasp 718 may be used in any orientation. Outer part 802 forms two fastener recesses 803(1) and 803(2) for receiving fasteners 806(1) and 806(2), as shown. Fasteners 806 each have a shaft 816 with a head 818 formed at one end and a thread 820 at the other end. In certain embodiments, head 818 is formed to couple only with a secure tool such that fasteners 806 are only operable with the security tool. In other embodiments, head 818 is formed to couple with a conventional tool, such as a small crosshead screwdriver. Within fastener recesses 803(1) and 803(2), outer part 802 forms two smaller apertures that allow shaft 816 and thread 820 of fastener 806 to pass but does not allow head 818 to pass. A C-clip 814 attaches to shaft 816 to retain fastener 806 within fastener recess 803. Inner part 804 forms two posts 808(1) and 808(2) that are sized and spaced to pass through holes 708 of strap 516. Each post 808(1) and 808(2) may have an insert 810(1) and 810(2) that receives thread 820 of corresponding fasteners 806(1) and 806(2) when outer part 802 is secured to inner part 804. In certain embodiments, inserts 810 are a threaded brass. In other embodiments, inserts 810 are each an unthreaded plastic tube and thread 820 cuts its own thread within the plastic tube.

In one example of operation, fasteners 806 are loosened (but retained by C-clips 814) and outer part 802 is removed from inner part 804. Strap 516 is positioned around a wrist or ankle of the test subject such that skin-side surface 512 of main body 514 is held against the skin of the test subject and strap portions 516A and 516B are overlapped. Inner part 804 is position inside strap 516 at the overlap and posts 808 are passed through holes 708 of both strap portions 516A and 516B. Outer part 802 is positioned over inner part 804 and fasteners 806 are screwed into inserts 810 to retain outer part 802 with inner part 804. Since posts 808 are passed through holes 708 of both strap portions 516A and 516B, clasp 718 retains wearable device 500 on the wrist or ankle of the test subject. In embodiments where fasteners 806 couple with a security tool, once applied to the wrist or ankle of the test subject, wearable device 500 may be removed using the security tool, or by cutting or breaking strap 516. Since strap portions 516A and 516B are overlapped and held together, electrical continuity through strap 516 is maintained, and therefore control circuit 626 may detect changes in electrical continuity when strap 516 is cut or when clasp 718 is loosened or removed.

In one example of operation, control circuit 326/626 includes a schedule for performing substance abuse readings. Accordingly, control circuit 326/626 measures the current generated by substance detection sensor 328/628 to determine a level of the abused substance based on the schedule. In another example of operation, control circuit 326/626 receives a message indicating that a substance abuse reading should be taken from relay device 106. Accordingly, control circuit 326/626 measures the current generated by substance detection sensor 328/628 to determine a level of the abused substance in response to the received message.

### Alternative Bottom Housing Design

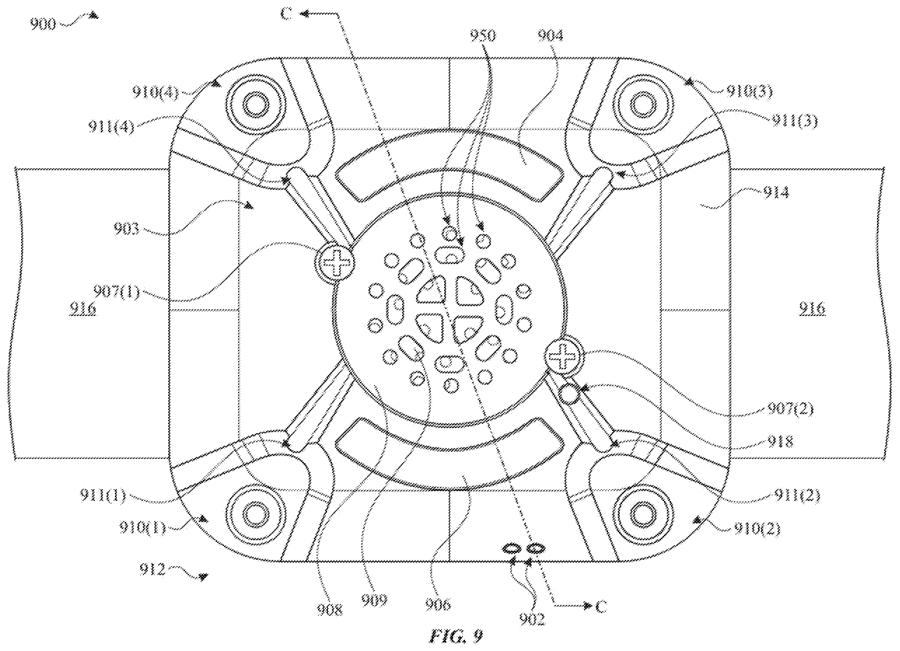

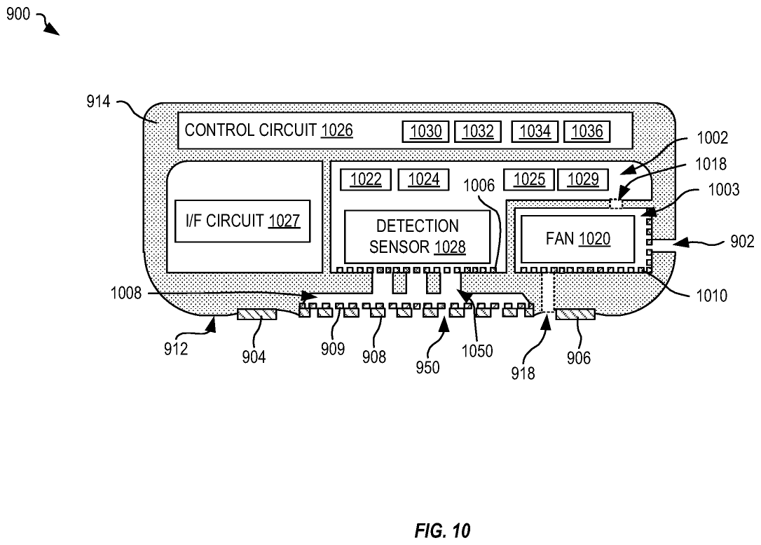

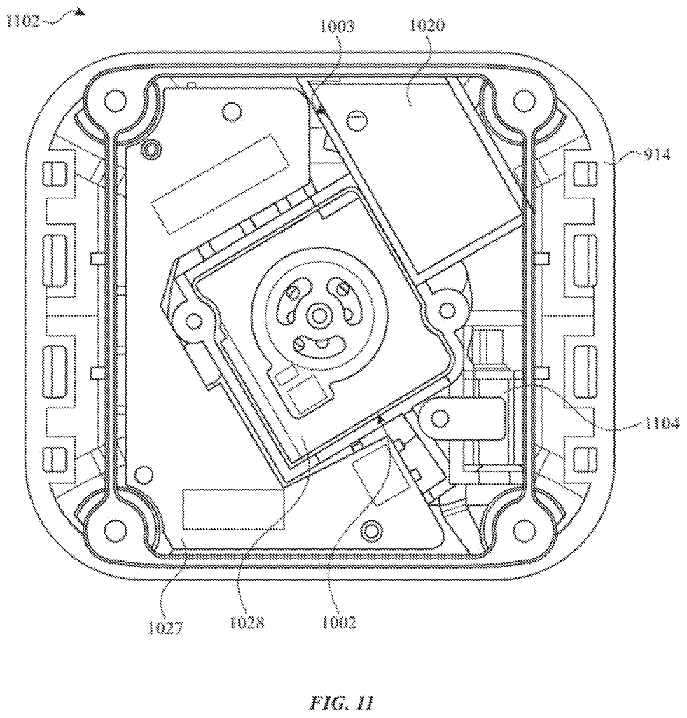

FIGS. 9 and 10 are schematics showing one example wearable device 900 that is similar to wearable devices 200 and 500 of FIGS. 2, 3, 5 and 6, but featuring an alternative channel design and internal component positioning, in embodiments. Wearable device 900 may represent wearable device 102 of FIG. 1. FIG. 9 is a schematic showing a bottom view of a main body 914 of wearable device 900 and FIG. 10 is a cross section C-C through main body 914 of FIG. 9. FIG. 11 shows an internal view of a bottom portion 1102 of wearable device 900 of FIGS. 9 and 10 where a top portion of main body 914 has been removed, in embodiments. FIGS. 9, 10, and 11 are best viewed together with the following description.

Wearable device 900 may include a time display 702 (see FIG. 7A) and associated time mechanism (e.g., mechanical, or digital) and may have a port for coupling with a charging cord 704; however, other embodiments of wearable device 900 may use wireless charging without departing from the scope hereof. A bottom surface 903 of main body 914 includes two arced skin contacts 904 and 906, a recessed area 1008, four corner recessed areas 910, and four channels 911. A dirt membrane 908 and a face plate 909 are mounted, using screws 907(1) and 907(2) for example, to cover recessed area 1008. Skin contacts 904, 906, and face plate 909 may be metal (e.g., grade 5 titanium, known as industrial titanium). Advantageously, test subjects using wearable device 900 do not have a reaction when titanium is used for skin contacts 904, 906, and face plate 909. Skin contacts 904 and 906 conduct electricity. Face plate 909 includes a plurality of apertures 950 and is positioned and retained over recessed area 1008 by at least one screw, making face plate 909 removable. Apertures 950 may be circular and/or elongated to prevent clogging. Recessed area 1008 and an internal chamber 1002 of main body 914 are fluidly coupled via one or more internal apertures 1050. One or more internal apertures 1050 may be oblong and/or curved to resist clogging. Dirt membrane 908 (e.g., an ingress protection (IP)65 membrane), positioned on face plate 909, operates to prevent ingress of dirt into recessed area 1008 and further into internal chamber 1002. Internal chamber 1002 may also include a water membrane 1006 (e.g., an IP68 membrane) positioned adjacent recessed area 1008 to exclude water from internal chamber 1002. Dirt membrane 908 and water membrane 1006 are air-permeable.

When wearable device 900 is worn, recessed area 1008 forms an airspace adjacent to the test subject's skin, from where air flows through substance detection sensor 1028 positioned within internal chamber 1002 via apertures 950, dirt membrane 908 and water membrane 1006. For example, natural movement of the test subject causes device 900 to move (e.g., rock) against the test subject's skin, whereby the rocking movement may cause air (e.g., a transdermal sample from the test subject) within recessed area 1008 to flow into chamber 1002. Recessed area 1008 is sized and shaped such that an airspace within recessed area 1008 allows the abused substance (e.g., alcohol) to pass through the test subject's skin into air within the airspace. For example, recessed area 1008 is circular with a depth of 3/64^{th} of an inch and a diameter of ¾ of an inch. Each channels 911 extends from a respective corner recess area 910 towards recessed area 1008. Corner recessed areas 910 and channels 911 provide airspaces adjacent to the test subject's skin that allow moisture to flow away from recessed area 1008.

Recessed area 1008, apertures 950, corner recessed areas 910, and channels 911 are a novel design that facilitate collection of air from above the skin of the test subject when device 900 is worn and also prevent the test subject's skin from blocking apertures 950 that would prevent air flow. For example, without recessed area 1008, corner recessed areas 910, and channels 911, the test subject's skin pressing against bottom surface 903, a skin-side surface 912, would prevent air flow through apertures 950 and into internal chamber 1002 where it is tested by substance detection sensor 1028.

Humidity in air flowing through device 900 adversely effects operation of substance detection sensor 1028 to detect the abused substance. Accordingly, skin-side surface 912 is designed to include corner recessed areas 910 and channels 911 that allow air not flowing through apertures 950 to disperse away from device 900, thereby reducing humidity between the test subject's skin and device 900. Advantageously, the amount of humidity that would otherwise collect and flow through apertures 950 is reduced, thereby reducing the adverse effects of humidity on substance detection sensor 1028.

Substance detection sensor 1028 electrically connects (connections not shown for clarity of illustration) with an control circuit 1026 also contained within main body 914. Control circuit 1026 may include one or more of: a processor 1030, memory 1032 storing machine-readable instructions executable by processor 1030, a global navigation satellite system (GNSS) receiver 1034, and a motion sensor 1036 (e.g., an accelerometer). Control circuit 1026 may include additional functionality including conditioning circuity, interface circuity, wireless communication circuity (e.g., Bluetooth, BLE, etc.), a watch display, and a battery. Control circuit 1026 may include other components without departing from the scope hereof. For example, control circuit 1026 may be implemented on one or more circuit boards. In certain embodiments, main body 914 includes a printed circuit board 1027 that implements an interface circuit that electrically interfaces one or more of fan 1020, heater 1022, pressure sensor 1024, temperature sensor 1025, and substance detection sensor 1028 with control circuit 1026. In certain embodiments, printed circuit board 1027 is implemented as a flex circuit. In certain embodiments, wearable device 900 may include a haptic generator 1104 that is controlled by control circuit 1026 to generates one of more haptic signals to attract the attention of the test subject. Haptic generator 1104 is for example a vibration motor.

A chemical reaction within substance detection sensor 1028 generates an electrical current when alcohol is present in the air flowing through substance detection sensor 1028, where an amount of the electrical current corresponds to an amount of the abused substance (e.g., alcohol) present in the air. Control circuit 1026 measures the electrical current from substance detection sensor 1028 to determine the amount of abused substance in the air. In one operational example, substance detection sensor 1028 operates continuously to detect levels as low as one molecule per 10 million molecules of air. In certain embodiments, substance detection sensor 1028 generates an electrical charge corresponding to the amount of abused substance in the air. Control circuit 1026 reads the electrical charge at intervals, such as once every twelve minutes when the level of the abused substance is below a threshold amount, and once every five minutes when the level of the abused substance is above the threshold amount. For example, a first reading may indicate a 0.10 mg/l of abused substance, a second reading may indicate a 0.12 mg/l of abused substance, and a next reading may indicate a 0.10 mg/l of abused substance, and so on. Such small variations is reading may be due to the small amounts of abused substance being detected. However, such small readings are indicative of substance abuse and are not resulting from environmental conditions.

Pressure sensor 1024 is positioned within internal chamber 1002 to detect air pressure within internal chamber 1002. In certain embodiments, wearable device 900 may include a heater 1022 (e.g., resistive heater) positioned within internal chamber 1002 that may be activated as needed to use pressure sensor 1024. Pressure sensor 1024 may be read at intervals to detect ambient air pressure. Accordingly, when control circuit 1026 determines that the air pressure is not changing, (e.g., by at least a predefined threshold amount), control circuit 1026 determines that apertures 950 may be blocked and control circuit 1026 sends an alert and/or notification to server 104 indicative of tampering with operation of device 900. Advantageously, control circuit 1026 detects when the test subject is attempting to compromise operation of wearable device 900 by blocking apertures 950 with a film or a gel, a paste, or other semi-liquid substance.

A temperature sensor 1025 may also be positioned within internal chamber 1002 to detect an internal temperature of wearable device 900 and thereby indicate when wearable device 900 is operating safely. Where temperature sensor 1025 indicates a temperature above a safety threshold, control circuit 1026 may determine that wearable device 900 has developed a fault, is not operating safely, and/or may pose a danger to the test subject. Accordingly, control circuit 1026 may stop operation of wearable device 900, such as by switching wearable device 900 off to prevent damage or harm.

In certain embodiments, wearable device 900 includes a fan 1020 (e.g., a micro fan) positioned in a second internal chamber 1003 that is independent of internal chamber 1002. Chamber 1003 has an air inlet 902 positioned on a side of main body 914 and an air outlet 918 positioned on skin-side surface 912 of main body 914. Chamber 1003 may also have a second air outlet 1018 into internal chamber 1002. When operating, fan 1020 draws air into second internal chamber 1003 via air inlet 902 and expels air from second internal chamber 1003 via air outlet 918 and second air outlet 1018. Second internal chamber 1003 may include a water membrane 1010 to prevent ingress of water via air inlet 902 and air outlet 918. In certain embodiments, water membrane 1010 is part of water membrane 1006. Water membrane 1010 is air-permeable. Fan 1020 may be activated as needed to move sweat away from recessed area 1008 (e.g., via channels 911 and corner recessed areas 910). Experimental use of wearable device 900 has shown that as the test subject perspires, perspiration doesn't have an opportunity to evaporate from beneath wearable device 900. Thus, when wearable device 900 is removed, moisture was felt on the skin covered by wearable device 900. This moisture appears to embody a certain amount of the abused substance that is neither being drawn in through substance detection sensor 1028 nor being evaporated. As wearable device 900 captures readings over one or more hours after first detecting the abused substance, substance detection sensor 1028 continues to detect presence of the abused substance even after the test subject's body has metabolized the abused substance. This continued detection of the abused substance is a false reading that may be referred to as a tale. Thus, it appears that the abused substance remains in the perspiration trapped beneath wearable device 900 after the test subject's body has metabolized the abused substance. When software with control circuit 1026 determines that the detected level of the abused substance is not dropping, control circuit 1026 activates fan 1020 to remove the perspiration beneath wearable device 900. In certain embodiments, wearable device 900 may include a humidity sensor 1029 within internal chamber 1002 that detects increased moisture levels within internal chamber 1002 (e.g., resulting from perspiration) and activates fan 1020 to reduce levels of moisture within internal chamber 1002 and beneath wearable device 900.

Dirt membrane 908 may be replaced or cleaned by removing face plate 909. Advantageously, replacement of dirt membrane 908 is relatively easy, whereas replacement of water membranes 1006 and 1010 involves opening main body 914. Advantageously, dirt membrane 908 may have some waterproofing functionality (e.g., blocking water from showering) and blocks dirt and debris from entering recessed area 1008, thereby protecting water membrane 1006. Although example IP ratings of 65 and 68 have been provided for dirt membrane 908, water membrane 1006, and water membrane 1010, other IP ratings may be used without departing from the scope hereof. Water membrane 1006/1010 rated at IP68 provides waterproofing at a depth of one meter for one minute, for example. Other IP ratings may be used where wearable device 900 is expected to operate in different environmental conditions.

### Power Save Function

Conventional GNSS locationing has limited accuracy (e.g., six meters) and also suffers from signal degradation due to atmospheric conditions and building effects for example. Thus, when the test subject and wearable device 900 is stationary, the captured GNSS location may drift. In an example where the test subject is stationary at home, a series of captured GNSS locations may include outliers such as a position in the kitchen, a position in the back yard, and a position in a neighbor's yard. Such outliers are typical in GNSS locations captured by wearable devices. Certain aspects of the present embodiments include the realization that this drift prevents detection of stationary conditions (e.g., when the test subject is asleep) using GNSS location data alone. The present embodiments solve this problem by using an algorithm that processes the GNSS location data and other sensor data (e.g., an accelerometer) together to detect when the test subject is changing location, and when the test subject is not changing location. Advantageously, by determining when the test subject is stationary, wearable device 900 may save battery power, such as by not determining GNSS locations, at least not a often as when the test subject is active. When other sensors indicate that the test subject is moving sufficiently to change location, wearable device 900 may start determining GNSS locations again. Although described with reference to wearable device 900, the described sleep function may be implemented within any of device 102 of FIG. 1, wearable device 200 of FIGS. 2 and 3, and wearable device 500 of FIGS. 5 and 6.

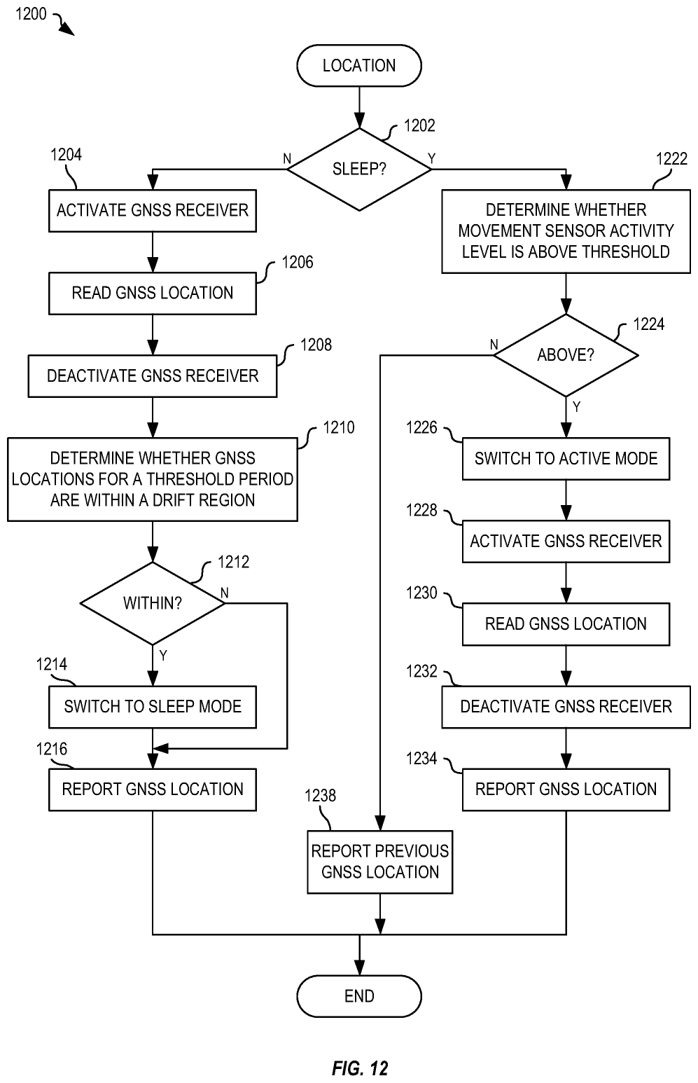

FIG. 12 is a flowchart illustrating one example sleep method 1200 for conserving battery power within a wearable device, in embodiments. Method 1200 may be implemented as machine readable instructions shored within memory 326/626/1026 of device 200/500/900 of FIGS. 2, 5, and 9, respectively. The following example uses the term sleep mode, however, this functionality may apply to any situation where the test subject is stationary, such as when watching TV, in the office, in bed sleeping, etc., and is not limited to only when the test subject is sleeping.

Block 1202 is a decision. If, in block 1202, method 1200 determines that a current location mode is sleep, method 1200 continues with block 1204; otherwise, method 1200 continues with block 1222. The current location mode is a two state variable stored in memory 322/522/1022 of control circuit 326/626/1026. Blocks 1204-1216 are performed when the current location mode indicates sleep and block 1222-1238 are performed when the current location mode does not indicate sleep (e.g., an active mode).

In block 1204, method 1200 activates the GNSS receiver. In one example of block 1204, control circuit 1026 activates GNSS receiver 1034. In block 1206, method 1200 reads the GNSS location from the GNSS receiver. In one example of block 1206, control circuit 1026 reads a current GNSS location from GNSS receiver 1034. In block 1208, method 1200 deactivates the GNSS receiver. In one example of block 1208, control circuit 1026 deactivates GNSS receiver 1034.

In block 1210, method 1200 determines whether GNSS locations for a threshold period and within a drift region. In one example of block 1210, control circuit 1026 determines whether GNSS locations captured within a predefined period (e.g., the last thirty minutes) are within a drift region (e.g., a circle of ten meter radius). Block 1212 is a decision. If, in block 1212, method 1200 determines, in block 1210, that the GNSS locations are within the drift region, method 1200 continues with block 1214; otherwise, method 1200 continues with block 1216.

In block 1214, method 1200 switches to sleep mode. In one example of block 1214, control circuit 326/626/1026 sets the two state variable stored in memory 322/522/1022 to indicate sleep mode. In block 1216, method 1200 reports the GNSS location. In one example of block 1216, control circuit 326/626/1026 stores the most recent GNSS location in memory 332/632/1032 and/or sends the more recent GNSS location in status message 110 to server 104 via relay device 106. Method then terminates until invoked to determine and report a subsequent location of wearable device 200/500/900.

In block 1222, method 1200 determines whether a movement sensor activity level is above a threshold level. In one example of block 1222, control circuit 1026 processes sensor data (e.g., accelerometer data, motion data, etc.) from motion sensor 1036 indicate activity is above a threshold level that indicates that the test subject is changing location. The activity threshold level is set at a level such that movement of wearable device 900 (e.g., arm movement of the test subject while asleep, sitting, or working) is below the activity threshold, whereas movement of wearable device 900 when the test subject stands and changes location (e.g., walks) is above the activity threshold. Block 1224 is a decision. If, in block 1224, method 1200 determines, from block 1222, that detected activity is above the activity threshold, method 1200 continues with block 1226; otherwise, method 1200 continues with block 1238.

In block 1226, method 1200 switches to active mode. In one example of block 1226, control circuit 326/626/1026 sets the two state variable stored in memory 322/522/1022 to indicate active mode (e.g., not sleep mode). In block 1228, method 1200 activates the GNSS receiver. In one example of block 1228, control circuit 1026 activates GNSS receiver 1034. In block 1230, method 1200 reads the GNSS location from the GNSS receiver. In one example of block 1230, control circuit 1026 reads a current GNSS location from GNSS receiver 1034. In block 1232, method 1200 deactivates the GNSS receiver. In one example of block 1232, control circuit 1026 deactivates GNSS receiver 1034. In block 1234, method 1200 reports the GNSS location. In one example of block 1234, control circuit 326/626/1026 stores the most recent GNSS location in memory 332/632/1032 and/or sends the more recent GNSS location in status message 110 to server 104 via relay device 106. Method then terminates until invoked to determine and report a subsequent location of wearable device 200/500/900.

In block 1238, method 1200 reports the previous GNSS location. In one example of block 1238, control circuit 326/626/1026 stores the most recent previously captured GNSS location in memory 332/632/1032 and/or sends the most recent previously captured GNSS location in status message 110 to server 104 via relay device 106. Advantageously, where the test subject is not changing location, wearable device 200/500/900 reports a static location that does not show drift. Without this sleep method 1200, other wearable devices would continue to show movement caused by drift. Advantageously, while the test subject is stationary (e.g., while sleeping for eight hours each day), wearable device 200/500/900 implements method 1200 to conserve battery power by not activating GNSS receiver during stationary periods.

In certain embodiments, wearable device 200/500/900 may indicate sleep mode within status message 110 such that server 104 learns that the test subject is stationary. Further, wearable device 200/500/900 may reduce the frequency of, or pause, subsequent status messages 110 sent to server 104 until wearable device 200/500/900 transitions back to active move. Accordingly, further battery power is saved by not having to activate the wireless transceiver within wearable device 200/500/900, particularly where the communication is cellular.

### Advanced GNSS Receiver

Where GNSS is implemented by the US Global Positioning System (GPS), where conventional GPS receiver chips receive and process only legacy frequency signals (L1 and L2) from GPS satellites. The L1 frequency carries a coarse/acquisition (C/A) code and both L1 and L2 frequency signals carry an encrypted Precision code (P(Y)-code) that is in quadrature with the C/A carrier. The C/A code and the P(Y) code are provided on the L1 frequency that signal allow conventional GPS positioning to operate by receiving only a single frequency (L1).

More recent development of the GPS introduced L2C signal that is transmitted on the L2 frequency. The L2C signal improves accuracy of navigation by providing an easy to track signal that may act as a redundant signal in case of localized interference. In certain embodiments, GNSS receiver 1034 is enhanced to receive both the L1 and L2 frequency signals simultaneously. Advantageously, the capture of two frequencies (L1 and L2) allows GNSS receiver 1034 to automatically correct for ionospheric delay, a cause of GPS error. Accordingly, where GNSS receiver 1034 uses both the L1 and L2 frequency signals, GNSS receiver 1034 reduces the amount of drift. Further, since the L2 frequency is lower than the L1 frequency signal, it penetrates clouds, trees, and buildings better than the L1 frequency. Accordingly, GNSS locationing within buildings is improved.

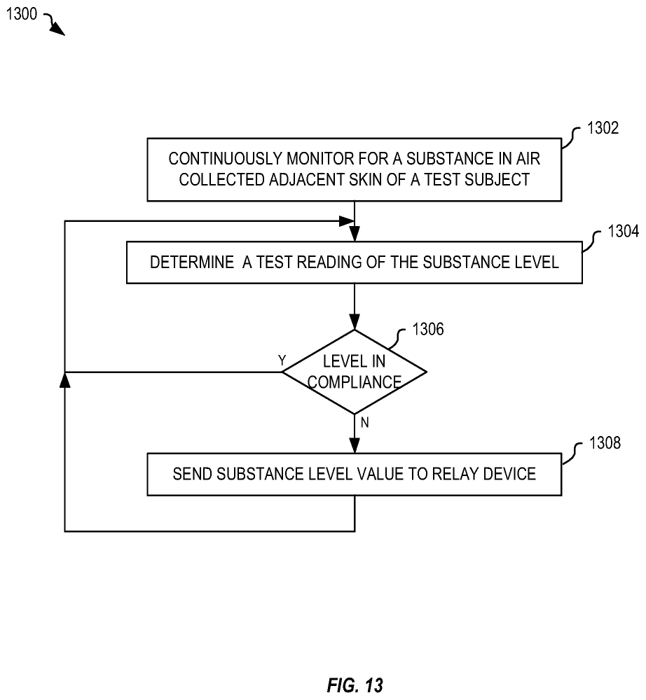

FIG. 13 is a flowchart showing one example substance sensing method 1300. Method 1300 detects a level of a substance and may be implemented as machine-readable instructions stored in memory 332/632/1032 and executed by processor 330/630/1030 of device 200/500/900 of FIGS. 3, 6, and 10, respectively.

In block 1302, method 1300 continuously monitors for a substance in air collected adjacent skin of a test subject. In one example of block 1302, channels 208 and 210 allow air adjacent a test subject's skin to naturally move, via apertures 250/252, through substance detection sensor 328, which continuously generates a current based on an amount of an abused substance in the air. In another example of block 1302, recessed areas 508 and 510 allow air adjacent a test subject's skin to naturally move, via apertures 550/552, through substance detection sensor 628, which continuously generates a current based on an amount of an abused substance in the air. In another example of block 1302, recessed area 1008 allows air adjacent a test subject's skin to naturally move, via apertures 950/951, through substance detection sensor 1028, which continuously generates a current based on an amount of an abused substance in the air. Particularly, substance detection sensor 328/628/1028 (e.g., a fuel cell) generates the current substantially continuously when the abused substance (e.g., alcohol) is within substance detection sensor 328/628/1028.

In block 1304, method 1300 determines a test reading of the substance level. In one example of block 1304, at a next scheduled sample time (e.g., at five-minute intervals, at twelve-minute intervals, etc.), control circuit 326/626/1026 senses the current from substance detection sensor 328/628/1028 and uses a formula to calculate a level (e.g., a substance level value) of substance within the air based on the sensed current. In another example of block 1304, control circuit 326/626/1028 senses the current from substance detection sensor 328/628/1028 and uses a lookup table to determine a level of substance within the air based on the sensed current.

Block 1306 is a decision. If, in block 1306, method 1300 determines that the substance level is in compliance, method 1300 continues with block 1304; otherwise, method 1300 continues with block 1308. For example, where the determined test reading of an abused substance indicates the substance level is below a predefined threshold, method 1300 returns to block 1304 to await the next sample time; however, where the test reading of the abused substance indicates the substance level is equal to or greater than the threshold value, method 1300 proceeds to block 1308. In another example of block 1306, where the determined test reading of a protective antiviral is at or above a safe minimum level, method 1300 continues with block 1304; however, when the determined test reading of a protective antiviral is below the safe minimum level, method 1300 continues with block 1308.

In block 1308, method 1300 sends the determined substance level to the relay device. In one example of block 1308, control circuit 326/626/1026 sends status message 110 containing the determined substance level to relay device 106. Method 1300 then continues with block 1304.

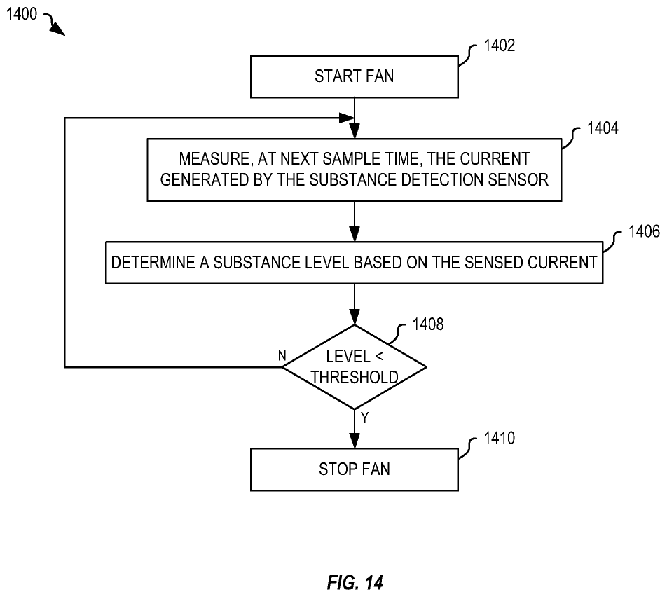

FIG. 14 is a flowchart illustrating one example method 1400 for removing residual substance from a substance detection sensor after a detected substance level has exceeded a threshold value. Method 1400 may be implemented as machine-readable instructions stored in memory 332/632/1032 and executed by processor 330/630/1030 of device 200/500/900 of FIGS. 3, 6, and 10, respectively. Method 1400 is invoked after the determined substance level was above the threshold value (e.g., in block 1308 of FIG. 13) to remove residual substance from substance detection sensor 328/628/1028. In one embodiment, method 1400 is invoked a certain period (e.g., 1 hour, 2 hours) after the first occurrence of the substance level exceeding the threshold level. In another embodiment, after the substance level exceeds the threshold value, method 1400 is invoked when the determined substance level subsequently drops below a second threshold value.

In block 1402, method 1400 starts a fan. In one example of block 1402, control circuit 326/626 activates fan 320/620 within chamber 302/602 to move air through substance detection sensor 328/628 via apertures 250/550 and 252/552, respectively. In another example of block 1402, control circuit 1026 activates fan 1020 within chamber 1003 to move air in through air inlet 902 and out through air outlet 918 to move perspiration away from recessed area 1008. In block 1404, method 1400 senses, at a next sample time, the current generated by the substance detection sensor. In one example of block 1404, at a next scheduled sample time (e.g., at one-minute intervals, at five-minute intervals, etc.), control circuit 326/626/1026 senses the current from substance detection sensor 328/628/1028. In block 1406, method 1400 determines a substance level based on the sensed current. In one example of block 1406, control circuit 326/626/1026 uses a formula to calculate a level of substance within the air based on the sensed current. In another example of block 1406, control circuit 326/626/1026 uses a lookup table to determine a level of substance within the air based on the sensed current.

Block 1408 is a decision. If, in block 1408, method 1400 determines that the substance level is less than a second threshold value, method 1400 continues with block 1410; otherwise, method 1400 continues with block 1404. In certain embodiments, block 1408 may also include a decision based on a time limit, whereby method 1400 proceeds to block 1410 after the fan has operated for the time limit, even though the substance level is not below the second threshold value. In block 1410, method 1400 stops the fan. In one example of block 1410, control circuit 326/626/1026 deactivates fan 320/620/1020.

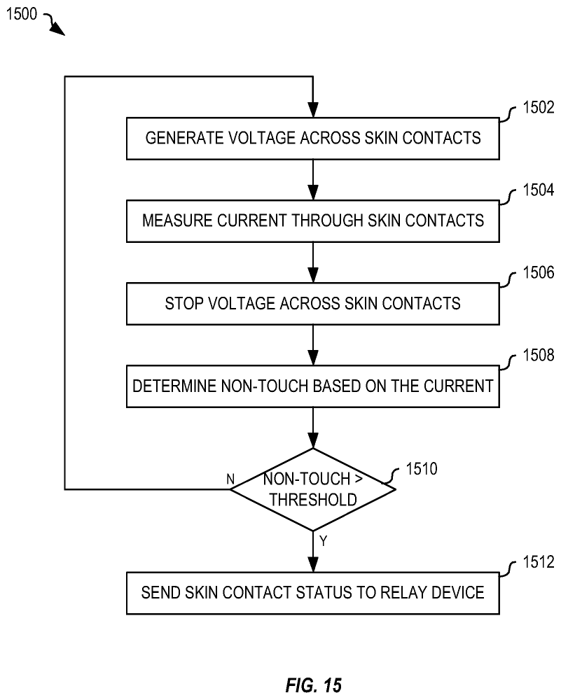

FIG. 15 is a flowchart illustrating one example method 1500 for detecting when operation of device 200/500/900 of FIGS. 2, 5, and 9, respectively, is compromised. Method 1500 may be implemented as machine-readable instructions stored in memory 332/632.1032 and executed by processor 330/630/1030 of device 200/500/900 of FIGS. 3, 6, and 9, respectively.

In block 1502, method 1500 generates a voltage across skin contacts. In one example of block 1502, control circuit 302/626/1026 applies a small voltage across skin contacts 204/504/904 and 206/506/906. In block 1504, method 1500 measures a current through the skin contacts. In one example of block 1504, control circuit 326/626/1026 measures a current flowing through the skin between skin contacts 204/504/904 and 206/506/906. In block 1506, method 1500 stops the voltage across the skin contacts. In one example of block 1506, control circuit 326/626/1026 stops applying the small voltage across skin contacts 204/504/904 and 206/506/906. In block 1508, method 1500 determines non-touch based on the current. In one example of block 1508, when control circuit 326/626/1026 determines that the measured current is outside the expected range, control circuit 326/626/1026 determines that device 200/500/900 has been removed from the test subject's skin and/or a film has been placed between the test subject's skin and device 200/500/900.

Block 1510 is a decision. If, in block 1510, method 1500 determines that the non-touch is greater than a non-touch threshold, method 1500 continues with block 1512; otherwise, method 1500 continues with block 1502. In one example of block 1510, control circuit 326/626/1026 determines that the period (e.g., number of samples taken) of deviation of the current from the expected range is greater than a threshold period. In block 1512, method 1500 sends skin contact status to relay device. In one example of block 1512, control circuit 326/626/1026 sends message 110 indicating the non-touch status detected by skin contacts 204/504/904 and 206/506/906 to relay device 106. Method 1500 may repeat at intervals (e.g., 1 second, 5 seconds, 10 seconds, etc.).

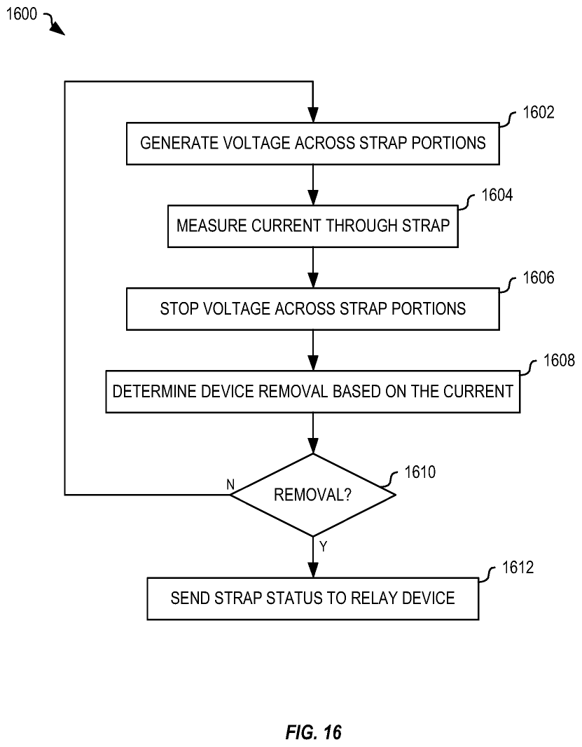

FIG. 16 is a flowchart illustrating one example method 1600 for detecting when device 200/500/900 of FIGS. 2, 5, and 9, respectively, is removed. Method 1600 may be implemented as machine-readable instructions stored in memory 332/632/1032 and executed by processor 330/630/1030 of device 200/500/900 of FIGS. 3, 6, and 10, respectively.

In block 1602, method 1600 generates a voltage across the strap portions of device 200/500/900 of FIGS. 2, 5, and 9, respectively. In one example of block 1602, control circuit 326/626/1026 applies a small voltage to either end of straps 116/516/916 (e.g., where strap portions 516A and 516B connect to main body 514 as shown in FIGS. 7A and 7B) such that a small electrical current passes through strap 116/516/916 when the ends of strap 116/516/916 are secured together (e.g., when clasp 718 secures the distal ends of strap 516 together). In block 1604, method 1600 measures a current through the strap. In one example of block 1604, control circuit 326/626/1026 senses the small current through strap 116/516/916. In block 1606, method 1600 stops the voltage across the strap portions. In one example of block 1606, control circuit 326/626/1026 stops applying the voltage across strap 116/516/916. In block 1608, method 1600 determine device removal based on the sensed current. In one example of block 1608, control circuit 326/626/1026 determines that device 200/500/900 has been removed when the sensed current is below a defined range, such as when strap 116/516/916 is cut or when the clasp (e.g., clasp 718) is loosened or opened. In another example of block 1608, control circuit 326/626/1026 detects tampering with device 200/500/900 when the sensed current is above the defined range, such as when someone attempts to bypass the current through strap 116/516/916.

Block 1610 is a decision. If, in block 1610, method 1600 determines that the device has been loosened or removed or that the strap is experiencing tampering, method 1600 continues with block 1612; otherwise, method 1600 continues with block 1602. In block 1612, method 1600 sends a strap status to the relay device. In one example of block 1612, control circuit 326/626/1026 sends message 110 indicating the strap status to relay device 106. Method 1600 may repeat at intervals (e.g., 1 second, 5 seconds, 10 seconds, etc.). In certain embodiments, block 1606 is omitted and blocks 1604, 1608, 1610, and 1612 repeat to monitor strap 516 substantially continuously.

### Annunciating Tracking Device

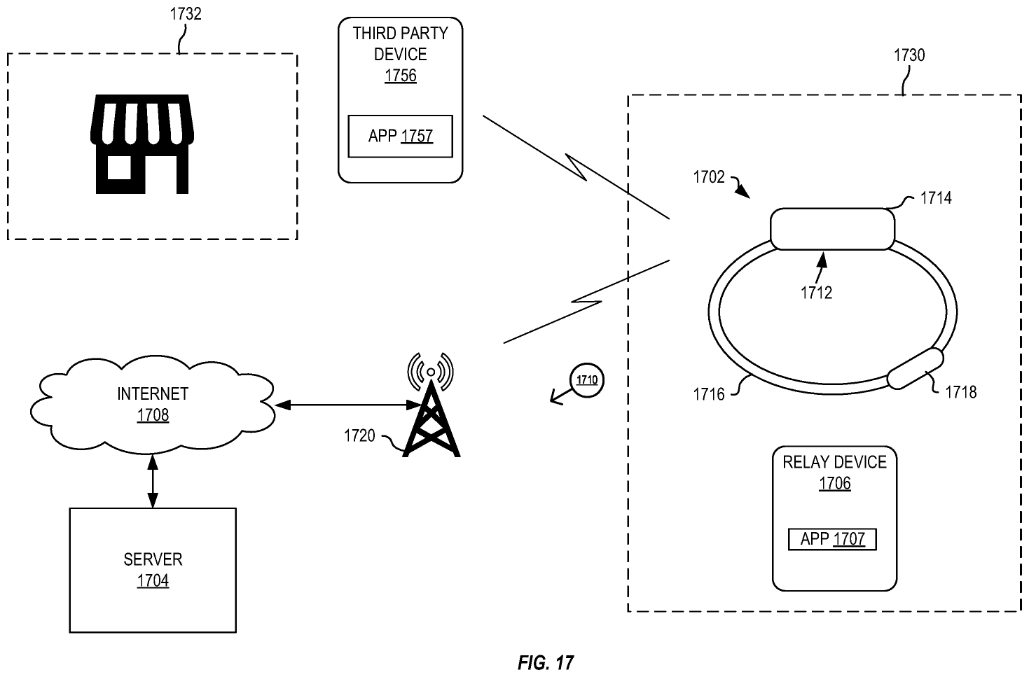

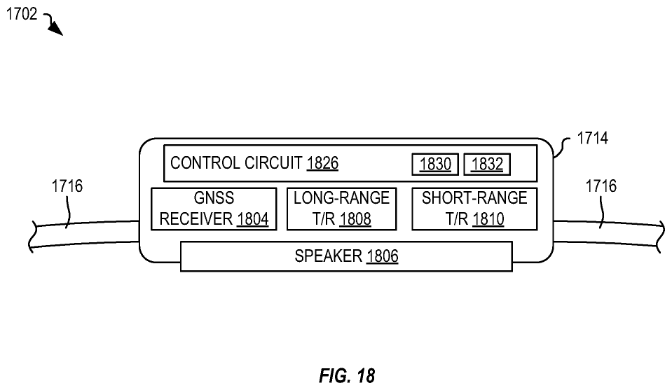

FIG. 17 is a schematic illustrating one example annunciating tracking device 1702 for monitoring movement of a person. FIG. 18 is a cross-sectional schematic diagram illustrating annunciating tracking device 1702 of FIG. 17 in further example detail. FIGS. 17 and 17 are best viewed together with the following description. Functionality of annunciating tracking device 1702 may be included with devices 200/500/900 of FIGS. 2, 5, and 9, respectively, in certain embodiments. For example, devices 200/500/900 may include one or more of long-range transceiver 1808 and short-range transceiver 1810 to allow one or more of cellular, Wi-Fi and Bluetooth communication.

Annunciating tracking device 1702 includes a main body 1714, a strap 1716, and a clasp 1718 that are similar to devices 200/500/900 of FIGS. 2, 5, and 9, respectively. However, a skin-side 1712 of device 1702 is different from skin-side surfaces 212/512/912 devices 200/500/900 in that device 1702 need not include apertures, recesses, or skin contacts when substance abuse is not monitored. Main body 1714 includes a GNSS receiver 1804, a speaker 1806, a long-range transceiver 1808 (e.g., a cellular transceiver with e-SIM, LORA, etc.), a short-range transceiver 1810 (e.g., Bluetooth, BLE, Wi-Fi, etc.) and a control circuit 1826. Control circuit 1826 includes a processor 1830 and memory 1832 storing machine-readable instructions that when executed by processor 1830 causes processor 1830 to implement functionality of device 1702 as described herein.

Control circuit 1826 includes a digital-to-analog converter and an amplifier that drive speaker 1806 to output audible sounds such as pre-recorded messages. Speaker 1806 may have an audio output of at least eighty decibels. Device 1702 may include other components and/or functions, such as a haptic device (e.g., a vibration generator) that may be activated to get the attention of the person wearing device 1702. Strap 1716 is substantially the same as straps 116 and 516 of devices 200 and 500 and device 1702 may detect when it is removed from a person's wrist or ankle. In certain embodiments, control circuit 1826 implements at least part of method 1600 of FIG. 16 to determine when strap 1716 is loosened, opened, cut, or otherwise tampered with. Where a relay device 1706 (e.g., a smartphone, a vehicle interface, a table computer, a laptop computer, etc.) is within wireless range of device 1702, control circuit 1826 uses short-range transceiver 1810 to sends the strap status to an app 1707 running on a relay device 106 as described for block 1612. However, where relay device 1706 is not available (e.g., not within wireless range or deactivated), control circuit 1826 sends the strap status to server 1704 using long-range transceiver 1808 to indicate that device 1702 is removed from the person's wrist or ankle. Using relay device 1706 is a preferred way of communicating with server 1704, since short-range transceiver 1810 uses less power than long-range transceiver 1808.

Conventionally, a cellular chip and SIM allows a device to connect with a single carrier. Accordingly, the device can communicate only when within communication range from a cell tower of that carrier. Advantageously, long-range transceiver 1808 includes multi-chip/SIM functionality that allows device 1702 to connect to nine different cell tower networks or carriers. Advantageously, device 1702 is not prevented from communicating because cell towers of any one carrier are not within communication range. Accordingly, device 1702 may connect with a cellular network that is closest and thereby achieve communications where single carrier other devices would fail to communicate. Further, by having more cell towers available, the closest may be selected such that transmission power may be reduced, thereby conserving battery power.

Further, short-range transceiver 1810 may be configured to connect to a Wi-Fi access point and communicate with server 104 through the Wi-Fi network and the Internet for example. In certain embodiments, control circuit 1826 may control short-range transceiver 1810 to connect with an open Wi-Fi network (e.g., a public hotspot) to communicate with server 104.

Control circuit 1826 activates GNSS receiver 1804 at intervals (e.g., one minute, five minutes, ten minutes, etc.) to determine a current location of device 1702. Control circuit 1826 may send the current location (shown as message 1710) to a server 1704 via a cellular communication provider 1720 and/or the Internet 1708. In certain embodiments, when no location violation is occurring, control circuit 1826 may buffer time tagged locations within memory 1832 and send the buffered information to server 1704 in a single message 1710 at greater intervals to reduce power used by long-range transceiver 1808 for communication with cellular communication provider 1720. However, where device 1702 determines a location violation has occurred, location information and/or notifications of the violation may be reported sooner (e.g., immediately) to server 1704.

Control circuit 1826 may store (e.g., in memory 1832) geographic coordinates for one or more of an inclusion zone, and one or more of an exclusion zone. The inclusion zone defines an area in which device 1702 (and person wearing device 1702) is to remain and should not leave. Each exclusion zone defines an area from which device 1702 (and person wearing device 1702) is excluded and should not enter. When control circuit 1826 determines, based on a current location of device 1702, that device 1702 has left the area defined by the inclusion zone or that device 1702 is within an exclusion zone, control circuit 1826 determine a location violation has occurred and outputs an annunciation via speaker 1806 with instruction for the person wearing device 1702 to correct the location violation.

Control circuit 1826 may also determine when it is near to a third-party device 1756 carried by a second person that that the person wearing device 1702 should not approach (e.g., a restraining order against the person approaching the second person). Third-party device 1756 (e.g., smartphone, tablet, etc.) runs an app 1757 that includes a unique identifier (e.g., unique to the second person), and the unique identifier is preloaded into memory 1832 of device 1702. When third-party device 1756 is in wireless communication range of short-range transceiver 1810, control circuit 1826 may communicate with app 1757 to receive a message that includes the unique identifier. When control circuit 1826 determines that the received unique identifier matches an entry in an identifier list within memory 1832, control circuit 1826 determine a location violation has occurred and outputs a prerecorded or synthesized message via speaker 1806 with instructions for the person wearing device 1702 to correct the location violation, such as “you are violating a restraining order, move away.” In certain embodiments, in response to determining the location violation, control circuit 1826 may send a message to third-party device 1756 instructing app 1757 to output a notification and/or an audible message indicating proximity of the person wearing device 1702 to the second person. Advantageously, the second person is immediately aware of the proximity of the person wearing device 1702.

In one example of operation, a person is required to remain within an inclusion zone 1730 defined by geographic coordinates (e.g., their home) that are stored in memory 1832. Device 1702 is attached to a wrist (or ankle) of the person and cannot be removed without device 1702 detecting the removal or tampering. Control circuit 1826 periodically (e.g., one minute, five minutes, ten minutes, etc.) activates GNSS receiver 1804 to determine its current location. When the person wearing device 1702 leaves the inclusion zone (e.g., their home), control circuit 1826 determines that its current location (e.g., at a next location determining period) is outside the defined area of the inclusion zone and drives speaker 1806 to output a prerecorded or synthesized messages with instructions for the person wearing device 1702 to correct the location violation, such as “you are leaving an inclusion zone, return now,” at a moderate volume. Control circuit 1826 may also use long-range transceiver 1808 to send the current location to server 1704. Where control circuit 1826 determines a subsequently determined location is outside the inclusion zone, control circuit 1826 drives speaker 1806 to output a prerecorded or synthesized messages with instructions for the person wearing device 1702 to correct the location violation, such as “you are outside the inclusion zone, return now,” at a loud volume.

In another example scenario, the person wearing device 1702 is prohibited from entering a certain store. Coordinates defining a boundary of the store as an exclusion zone 1732 are stored in memory 1832. When control circuit 1826 determines that a current location of device 1702 is near a boundary of exclusion zone 1732, control circuit 1826 may cause speaker 1806 to output, at a moderate volume, a prerecorded or synthesized message with instructions for the person wearing device 1702 to correct the location violation, such as “you are entering an exclusion zone, turn away.” When control circuit 1826 determines that a current location of device 1702 is within exclusion zone 1732, control circuit 1826 may cause speaker 1806 to output, at a loud volume, a prerecorded or synthesized message with instructions for the person wearing device 1702 to correct the location violation, such as “you are within an exclusion zone, leave now.”

Accordingly, device 1702 may provide an audible warning to remind the person wearing device 1702 of any zone and/or restrain violations and also provides a warning to other people of the location violation, since speaker 1806 may output the message at a loud volume (e.g., eighty decibels).

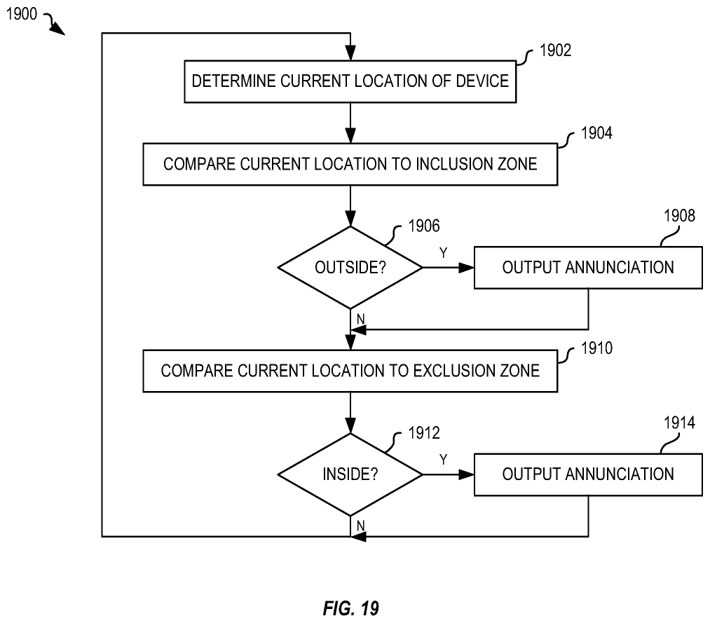

FIG. 19 is a flowchart illustrating one example method 1900 for detecting and announcing location violations by annunciating tracking device 1702 of FIG. 17. Method 1900 is implemented as machine-readable instructions stored in memory 1832 and executed by processor 1830 for example.

In block 1902, method 1900 determines a current location of the device. In one example of block 1902, at intervals, control circuit 1826 activates GNSS receiver 1804 to determine a current location based on satellite signals, and then deactivates GNSS receiver 1804 to save battery power. In block 1904, method 1900 compares the current location to an inclusion zone. In one example of block 1904, control circuit 1826 compares the current location to boundary coordinates of the inclusion zone defined within memory 1832 to determine whether the current location is outside the inclusion zone and a location violation has occurred. Block 1906 is a decision. If, in block 1906, method 1900 determines that the current location is outside the inclusion zone, method 1900 continues with block 1908; otherwise, method 1900 continues with block 1910.

In block 1908, method 1900 outputs an annunciation. In one example of block 1908, control circuit 1826 drives speaker 1806 to output a prerecorded or synthesized annunciation with instructions for the person wearing device 1702 to correct the location violation, such as “you are leaving an inclusion zone, return now.” Method 1900 then continues with block 1910.

In block 1910, method 1900 compares the current location to an exclusion zone. In one example of block 1910, control circuit 1826 compares the current location to boundary coordinates of each exclusion zone defined within memory 1832 to determine whether the current location is inside the exclusion zone and a location violation has occurred. Block 1912 is a decision. If, in block 1912, method 1900 determines that the current location is inside the exclusion zone, method 1900 continues with block 1914; otherwise, method 1900 continues with block 1902. In block 1914, method 1900 outputs an annunciation of the location violation. In one example of block 1914, control circuit 1826 drives speaker 1806 to output a prerecorded or synthesized annunciation with instructions for the person wearing device 1702 to correct the location violation, such as “you are entering an exclusion zone, exit now.” Method 1900 then continues with block 1902.

Method 1900 repeats at intervals to determine its current locations and then determine whether the current location indicates a violation of a restraint defined within memory 1832.

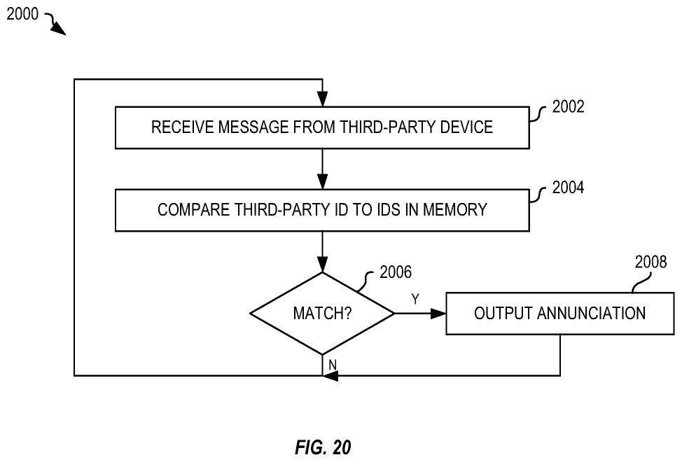

FIG. 20 is a flowchart illustrating one example method 2000 for detecting and announcing location violations by annunciating tracking device 1702 of FIG. 17. Method 2000 is implemented as machine-readable instructions stored in memory 1832 and executed by processor 1830 for example. Method 2000 is triggered by a communication with a third-party device, for example. Method 2000 operates independently of method 1900 and is driven by control circuit 1826 receiving a message from app 1757 via short-range transceiver 1810.

In block 2002, method 2000 waits to receive a message from a third-party device. In one example of block 2002, control circuit 1826 receives a message including a third-party unique identifier via short-range transceiver 1810 from third-party device 1756. In block 2004, method 2000 compares the third-party unique identifier to identifier in memory. In one example of block 2004, control circuit 1826 compares the third-party unique identifier received from third-party device 1756 to identifiers stored in memory 1832 to determine whether a location violation has occurred.

Block 2006 is a decision. If, in block 2006, method 2000 determines that the third-party unique identifier matches an entry within an identifier list stored in memory, method 2000 continues with block 2008; otherwise, method 2000 continues with block 2002. In block 2008, method 2000 outputs an annunciation of the location violation. In one example of block 2008, control circuit 1826 drives speaker 1806 to output a prerecorded or synthesized annunciation with instructions for the person wearing device 1702 to correct the location violation, such as “you are violating a restraining order, move away now.” Method 2000 then continues with block 2002.

Annunciating tracking device 1702 advantageously detects and annunciates location violations in a way that gets immediate attention of the person wearing device 1702 and of other people at or near that location.

Annunciating tracking device 1702 may implement the power saving functionality described above with reference to FIG. 12. For example, device 1702 may enter a sleep mode when stationary, such as when a test subject wearing device 1702 is asleep or watching TV. Further, device 1702 may implement the advanced GNSS receiver described above whereby GNSS receiver 1804 is enhanced to receive both the L1 and L2 frequency signals simultaneously. Advantageously, the capture of two frequencies (L1 and L2) allows GNSS receiver 1804 to automatically correct for ionospheric delay, a cause of GPS error. Accordingly, where GNSS receiver 1804 uses both the L1 and L2 frequency signals, GNSS receiver 1804 reduces the amount of drift.

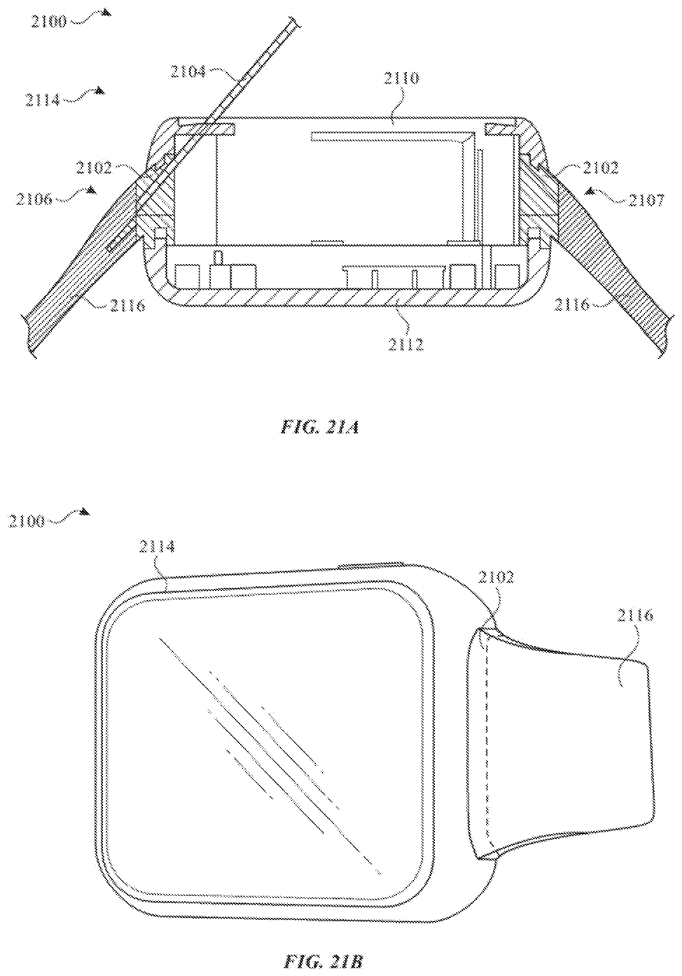

FIG. 21A is a cross-sectional diagram of a wearable device 2100 during manufacturer illustrating connection of a strap 2116 to a main body 2114 of a wearable device 2100, in embodiments. FIG. 21B is a perspective view showing connection of strap 2116 to main body 2114 of wearable device 2100 of FIG. 21A, in embodiments. FIGS. 21A and 21B are best viewed together with the following description. Wearable device 2100 may represent any of device 102, wearable device 200, wearable device 500, wearable device 900, and device 1702 of FIGS. 1, 2, 5, 9, and 17, respectively.

In certain embodiments, strap 2116 is a molded conductive silicon material. Main body 2114 is formed of a top portion 2110 and a bottom portion 2112. Each end 2106, 2107 of strap 2116 is mechanically keyed and over-molded with a strap connector 2102, which is also mechanically keyed and over-molded with top portion 2110 of main body 2114. Strap connector 2102 has a material composition that includes nylon, for example. Advantageously, over-molding top portion 2110 on to strap connectors 2102 provides a strong connection and a tight seal between strap 2116 and main body 2114 that prevents ingress of water, humidity, and dust into main body 2114.

In certain embodiments, a stainless steel wire 2104 is embedded into strap 2116 and passes through strap connector 2102 into main body 2114 to provide an electrical connection to a control circuit (not shown, e.g., one of control circuits 326, 626, 1026, and 1826 of FIGS. 3, 6, 10, and 18) that allows the control circuit to monitor for tampering of strap 2116, as described above. Although not shown, strap end 2107 may also include a stainless steel wire, similar to stainless steel wire 2104. Advantageously, the use of nylon strap connector 2102 overcomes the difficulty of over-molding ABS onto silicon.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

### Combination of Features

Features described above as well as those claimed below may be combined in various ways without departing from the scope hereof. The following enumerated examples illustrate some possible, non-limiting combinations:

(A1) A wearable device for detecting an abused substance, including: a main body forming an internal chamber and having a first recessed area and a second recessed area on a skin-side of the main body, the main body forming a plurality of first apertures between the first recessed area and the internal chamber; a substance detection sensor located within the internal chamber; a strap attached to the main body for securing the wearable device to a wrist or ankle of a test subject; and a control circuit including a processor and memory storing machine-readable instructions that, when executed by the processor, cause the control circuit to: determine a first substance level value from an output of the substance detection sensor indicative of a level of the abused substance in air flowing through the internal chamber; and send the first substance level value to an external server.

(A2) In embodiments of (A1), the strap is electrically conductive, the memory further includes machine-readable instructions that, when executed by the processor, cause the control circuit to: apply a voltage across the strap; monitor a current through the strap; and send a notification to a server when changes in the current indicate tampering or removal of the wearable device.

(A3) In either of embodiments (A1) or (A2), the strap including a first strap portion attached at one end to a first side of the main body and a second strap portion attached at one end to a second side, opposite the first side of the main body, wherein the voltage is applied at the main body across the first strap portion and the second strap portion.

(A4) Any of embodiments (A1)-(A3) further including a clasp having an inner part with a post and an outer part with a fastener that couples with the inner part, wherein the post passes through a first aperture formed by the first strap portion, through a second aperture formed by the second strap portion, and the first strap portion and the second strap portion are retained within the clasp when the outer part is secured to the inner part by the fastener.

(A5) In any of embodiments (A1)-(A4), the strap having a material composition that includes one or more of rubber, silicone, plastic, and polyurethane, in a formulation that incorporates a conductive element or compound, such as carbon black.

(A6) In any of embodiments (A1)-(A5), the first recessed area and the second recessed area are sized and shaped to allow the abused substance to transfer from skin of the test subject into the air within the first recessed area.

(A7) Any of embodiments (A1)-(A6) further including: a first pressure sensor located within the internal chamber; a second pressure sensor positioned within a sub-chamber formed by the main body and fluidly coupled with one of the first apertures and fluidically isolated from the internal chamber; and the memory further including machine-readable instructions that, when executed by the processor, cause the control circuit to: determine a first pressure value from an output of the first pressure sensor; determine a second pressure value from an output of the second pressure sensor; and send a notification to a server indicating that at least one of the plurality of first apertures is blocked when the second pressure value is different from the first pressure value by at least a threshold amount.

(A8) In any of embodiments (A1)-(A7), reading the first pressure sensor occurs at a first rate and reading the second pressure sensor occurs at a second rate faster than the first rate.

(A9) Any of embodiments (A1)-(A8) further including: a dirt membrane positioned at the plurality of first apertures that prevents ingress of dirt; and a water membrane positioned within the internal chamber that prevents ingress of water into the internal chamber; wherein the dirt membrane and the water membrane are air-permeable.

(A10) In any of embodiments (A1)-(A9), the main body forming a second aperture between the second recessed area and the internal chamber.

(A11) In any of embodiments (A1)-(A10), the second recessed area being formed at an outer edge of the skin-side of the main body.

(A12) In any of embodiments (A1)-(A11), the second recessed area is sized and shaped to allow the air to move from the internal chamber into an external environment.

(A13) Any of embodiments (A1)-(A12) further including at least one channel formed on the skin-side of the main body from the second recessed area toward the first recessed area.

(A14) In any of embodiments (A1)-(A13), the first recessed area and the second recessed area are sized and shaped to reduce humidity at the skin-side of the main body.

(A15) Any of embodiments (A1)-(A14) further including a fan located within the internal chamber, the memory further including machine-readable instructions that, when executed by the processor, cause the control circuit to operate the fan to move the air through the substance detection sensor to clear residual substance.

(A16) In any of embodiments (A1)-(A15), the memory further including machine-readable instructions that, when executed by the processor, cause the control circuit to determine, at intervals, a second substance level value from the output of the substance detection sensor and stop operating the fan when the second substance level value falls below a threshold value.

(A17) In any of embodiments (A1)-(A16) further including a global navigation satellite system (GNSS) receiver, the memory further storing machine-readable instructions that, when executed by the processor, cause the control circuit to read a current location from the GNSS receiver and report the current location with the first substance level value.

(A18) In any of embodiments (A1)-(A17), the GNSS receiver receiving both L1 and L2 frequency signals to automatically correct for ionospheric delay.

(A19) In any of embodiments (A1)-(A18), the memory further storing machine-readable instructions that, when executed by the processor, cause the control circuit to transition to a sleep mode when the wearable device is stationary to conserve battery power, wherein the processor does not activate the GNSS receiver when in sleep mode and reports a last determined GNSS location with the first substance level value.

(A20) In any of embodiments (A1)-(A19) further including a strap connector that is over-molded onto a mechanically keyed end of the strap and a top portion of the main body is over-molded onto a keyed portion of the strap connector.

(A21) In any of embodiments (A1)-(A20), the strap connector has a material composition that includes nylon to prevent ingress of water, humidity, and dust into the main body.

(B1) A method for detecting substance compliance using a wearable device positioned on a wrist or ankle of a test subject, including: continuously detecting, by a substance detection sensor located in an internal chamber of the wearable device, a substance within air from a first recessed area, located between a skin-side of the wearable device and skin of the test subject, via a first aperture formed between the internal chamber and the first recessed area; determining a substance level value from an output of the substance detection sensor; and sending the substance level value to an external server when the substance level value indicates a level of the abused substance that is not in compliance.

(B2) In embodiments of (B1), said sending including sending the substance level to the external server via a relay device.

(B3) Either of embodiments (B1) or (B2) further including: after a predefined period since determining the substance level value is not in compliance, activating a fan positioned within a chamber of a main body of the wearable device; determining, at intervals, a second substance level value from the output of the substance detection sensor; and deactivating the fan when the second substance level value is below a second predefined threshold value.

(B4) Any of embodiments (B1)-(B3) further including: applying a voltage across two different ends of a strap of the wearable device; monitoring a current through the strap; detecting changes in the current indicative of tampering; and sending a notification indicative of the tampering to the external server.

(B5) Any of embodiments (B1)-(B4) further including: determining a first pressure value from an output of a pressure sensor located in the internal chamber; and sending a notification to the external server indicating that the at least one first aperture is blocked by a foreign substance when at least one subsequently read second pressure value differs from the first pressure value by less than at least a threshold amount.

(B6) Any of embodiments (B1)-(B5) further including: reading a first pressure value from a pressure sensor included in the internal chamber; reading a second pressure value from a second pressure sensor positioned within a sub-chamber fluidly coupled with one of the at least one first aperture and not fluidly coupled to the internal chamber; and sending a notification to the external server indicating that the at least one first aperture is blocked when the second pressure value differs from the first pressure value by at least a threshold amount.

(B7) Any of embodiments (B1)-(B6) further including sending a status message to the external server when the substance level value is in compliance, the status message including a battery level of the wearable device to indicate operability of the wearable device.

(B8) Any of embodiments (B1)-(B7) further including: determining that the wearable device is stationary when global navigation satellite system (GNSS) locations read within a predefined period from a GNSS receiver of the wearable device are within a drift region; and transitioning the wearable device to a sleep mode in which a last read GNSS location is sent to the external server.

(B9) Any of embodiments (B1)-(B8) further including: determining that the wearable device is changing location when motion data from a motion sensor within the wearable device is greater than a motion threshold; and transitioning the wearable device out of the sleep mode, wherein a current location determined from the GNSS receiver is sent to the external server.

(C1) An annunciating tracking device, including: a main body; a strap coupled with the main body; a clasp for securing the strap to itself to attach the annunciating tracking device to a test subject; a speaker located with the main body; and a control circuit positioned within the main body and having: a global navigation satellite system (GNSS) receiver; a long-range transceiver; a processor; and memory storing (a) one or more of first geographic coordinates defining an inclusion zone and second geographic coordinates defining an exclusion zone, and (b) machine-readable instructions that when executed by the processor cause the control circuit to: activate, at first intervals, the GNSS receiver to determine a current location of the annunciating tracking device; determine a location violation when the current location is outside the inclusion zone or the current location is within the exclusion zone; and output, via the speaker, a prerecorded or synthesized message with instructions for a person wearing the annunciating tracking device to correct the location violation.

(C2) In embodiments of (C1), the memory further storing machine-readable instructions that, when executed by the processor, cause the control circuit to send, using the long-range transceiver, a notification of the location violation to a server.

(C3) In either of embodiments (C1) or (C2), the long-range transceiver implementing a protocol selected from the group consisting of cellular and LORA.

(C4) Any of embodiments (C1)-(C3) further including a short-range transceiver, the memory further storing machine-readable instructions that, when executed by the processor, cause the control circuit to: receive a message including a unique identifier from a third-party device; and determine the location violation when the unique identifier matches an entry in an identifier list stored in the memory.

(C5) In any of embodiments (C1)-(C4), the short-range transceiver implementing a Bluetooth protocol.

(C6) In any of embodiments (C1)-(C5), the memory further storing machine-readable instructions that, when executed by the processor, cause the control circuit to send, using the short-range transceiver, a notification of the location violation to a server via a relay device when the relay device is in wireless range of the annunciating tracking device.

(C7) In any of embodiments (C1)-(C6), the GNSS receiver receiving both L1 and L2 frequency signals to automatically correct for ionospheric delay.

(C8) In any of embodiments (C1)-(C7), the memory further storing machine-readable instructions that, when executed by the processor, cause the control circuit to transition to a sleep mode when the annunciating tracking device is stationary to conserve battery power, wherein the processor does not activate the GNSS receiver when in the sleep mode.

(C9) Any of embodiments (C1)-(C8) further including a strap connector that is over-molded onto a mechanically keyed end of the strap and a top portion of the main body is over-molded onto a keyed portion of the strap connector.

(C10) In any of embodiments (C1)-(C9), the strap connector having a material composition that includes nylon to prevent ingress of water, humidity, and dust into the main body.

What is claimed is:

1. A wearable device for detecting an abused substance, comprising:
a main body forming an internal chamber and having a first recessed area and a second recessed area on a skin-side of the main body, the main body forming a plurality of first apertures between the first recessed area and the internal chamber;
a substance detection sensor located within the internal chamber;
a strap attached to the main body for securing the wearable device to a wrist or ankle of a test subject; and
a control circuit including a processor and memory storing machine-readable instructions that, when executed by the processor, cause the control circuit to:
determine a first substance level value from an output of the substance detection sensor indicative of a level of the abused substance in air flowing through the internal chamber; and
send the first substance level value to an external server.

2. The wearable device of claim 1, wherein the strap is electrically conductive, the memory further comprising machine-readable instructions that, when executed by the processor, cause the control circuit to:
apply a voltage across the strap;
monitor a current through the strap; and
send a notification to a server when changes in the current indicate tampering or removal of the wearable device.

3. The wearable device of claim 2, the strap comprising a first strap portion attached at one end to a first side of the main body and a second strap portion attached at one end to a second side, opposite the first side of the main body, wherein the voltage is applied at the main body across the first strap portion and the second strap portion.

4. The wearable device of claim 1, further comprising:
a first pressure sensor located within the internal chamber;
a second pressure sensor positioned within a sub-chamber formed by the main body and fluidly coupled with one of the first apertures and fluidically isolated from the internal chamber; and
the memory further comprising machine-readable instructions that, when executed by the processor, cause the control circuit to:
determine a first pressure value from an output of the first pressure sensor;
determine a second pressure value from an output of the second pressure sensor; and
send a notification to a server indicating that at least one of the plurality of first apertures is blocked when the second pressure value is different from the first pressure value by at least a threshold amount.

5. The wearable device of claim 1, the main body forming a second aperture between the second recessed area and the internal chamber.

6. The wearable device of claim 1, the second recessed area being formed at an outer edge of the skin-side of the main body, wherein the second recessed area is sized and shaped to allow the air to move from the internal chamber into an external environment.

7. The wearable device of claim 1, wherein the first recessed area and the second recessed area are sized and shaped to reduce humidity at the skin-side of the main body.

8. The wearable device of claim 1, further comprising a fan located within the internal chamber, the memory further comprising machine-readable instructions that, when executed by the processor, cause the control circuit to operate the fan to move the air through the substance detection sensor to clear residual substance.

9. The wearable device of claim 8, the memory further comprising machine-readable instructions that, when executed by the processor, cause the control circuit to determine, at intervals, a second substance level value from the output of the substance detection sensor and stop operating the fan when the second substance level value falls below a threshold value.

10. The wearable device of claim 1, further comprising a global navigation satellite system (GNSS) receiver, the memory further storing machine-readable instructions that, when executed by the processor, cause the control circuit to:
read a current location from the GNSS receiver and report the current location with the first substance level value; and
transition to a sleep mode when the wearable device is stationary to conserve battery power, wherein the processor does not activate the GNSS receiver when in sleep mode and reports a last determined GNSS location with the first substance level value.

11. The wearable device of claim 1, further comprising a strap connector that is over-molded onto a mechanically keyed end of the strap and a top portion of the main body is over-molded onto a keyed portion of the strap connector.

12. The wearable device of claim 11, wherein the strap connector has a material composition that includes nylon to prevent ingress of water, humidity, and dust into the main body.

13. A method for detecting substance compliance using a wearable device positioned on a wrist or ankle of a test subject, comprising:
continuously detecting, by a substance detection sensor located in an internal chamber of the wearable device, a substance within air from a first recessed area, located between a skin-side of the wearable device and skin of the test subject, via a first aperture formed between the internal chamber and the first recessed area;
determining a substance level value from an output of the substance detection sensor; and
sending the substance level value to an external server when the substance level value indicates a level of the substance that is not in compliance.

14. The method of claim 13, further comprising:
after a predefined period since determining the substance level value is not in compliance, activating a fan positioned within a chamber of a main body of the wearable device;
determining, at intervals, a second substance level value from the output of the substance detection sensor; and
deactivating the fan when the second substance level value is below a second predefined threshold value.

15. The method of claim 13, further comprising:
applying a voltage across two different ends of a strap of the wearable device;
monitoring a current through the strap;
detecting changes in the current indicative of tampering; and
sending a notification indicative of the tampering to the external server.

16. The method of claim 13, further comprising:
determining a first pressure value from an output of a pressure sensor located in the internal chamber; and
sending a notification to the external server indicating that the first aperture is blocked by a foreign substance when at least one subsequently read second pressure value differs from the first pressure value by less than at least a threshold amount.

17. The method of claim 13, further comprising:
reading a first pressure value from a pressure sensor included in the internal chamber;
reading a second pressure value from a second pressure sensor positioned within a sub-chamber fluidly coupled with one of the first aperture and not fluidly coupled to the internal chamber; and
sending a notification to the external server indicating that the first aperture is blocked when the second pressure value differs from the first pressure value by at least a threshold amount.

18. The method of claim 13, further comprising sending a status message to the external server when the substance level value is in compliance, the status message including a battery level of the wearable device to indicate operability of the wearable device.

19. The method of claim 13, further comprising:
determining that the wearable device is stationary when global navigation satellite system (GNSS) locations read within a predefined period from a GNSS receiver of the wearable device are within a drift region; and
transitioning the wearable device to a sleep mode in which a last read GNSS location is sent to the external server.

20. The method of claim 19, further comprising:
determining that the wearable device is changing location when motion data from a motion sensor within the wearable device is greater than a motion threshold; and
transitioning the wearable device out of the sleep mode, wherein a current location determined from the GNSS receiver is sent to the external server.
